(19) United States
(12) Patent Application Publication
(10) Pub. No.: US 2008/0146593 A1
(43) Pub. Date: Jun. 19, 2008

(54) Substituted 5-Phenyl Pyrimidines I In Therapy

(75) Inventors: Joachim Rheinheimer, Ludwigshafen (DE); Thomas Grote, Wachenheim (DE); Bernd Muller, Frankenthal (DE); Barbara Nave, Deidesheim (DE); Frank Schieweck, Hessheim (DE); Anja Schwogler, Mannheim (DE); Thorsten Jabs, Hassloch (DE); Carsten Blettner, Hong Kong (CN)

Correspondence Address: CONNOLLY BOVE LODGE & HUTZ, LLP, P O BOX 2207, WILMINGTON, DE, 19899, US

(73) Assignee: Basf Aktiengesellschaft, Ludwigsshafen (DE)

(21) Appl. No.: 11/815,042

(22) PCT Filed: Jan. 30, 2006
(86) PCT No.: PCT/EP06/00774

§ 371 Date: Jul. 30, 2007

(30) Foreign Application Priority Data
Jan. 31, 2005 (EP) 05001955.3

(51) Int. Cl. A61K 31/505; A61K 31/513; A61P 35/00

(52) U.S. Cl. 514/269; 514/256

(57) ABSTRACT

The present invention relates to substituted 5-phenyl pyrimidines I, which carry a radical X in the 4-position of the pyrimidine ring, a radical Y in the 6-position of the pyrimidine ring, the radical X denoting a group of the formula NR¹R², OR^{1a} or SR^{1a}, in which R¹, R², independently of each other, denote hydrogen, C₁-C₁₀-alkyl, C₂-C₆-alkenyl, C₂-C₆-alkynyl, C₁-C₁₀-haloalkyl, C₃-C₈-cycloalkyl, C₃-C₈-halocycloalkyl, phenyl, or 5- or 6-membered heteroaryl or 5- or 6-membered heterocyclyl, containing 1, 2, 3 or 4 nitrogen atoms or 1, 2 or 3 nitrogen atoms and one sulfur or oxygen atom as ring members, which radicals may be unsubstituted or may carry 1, 2, 3 or 4 radicals R^{a1}; or the radical NR¹R² may also form a 5- or 6-membered optionally substituted heterocyclic ring, containing 1, 2, 3 or 4 nitrogen atoms or 1, 2 or 3 nitrogen atoms and one sulfur or oxygen atom as ring members, which are non-adjacent to the nitrogen of NR¹R², in which two adjacent C atoms or one N atom and one adjacent C atom can be linked by a C₁-C₄-alkylene chain and wherein the heterocyclic ring may be unsubstituted or may carry 1, 2, 3 or 4 radicals R^{a1} as defined in claim 1, R^{1a} has one of the meanings given for R¹ except for hydrogen; the radical Y being selected from the group consisting of halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the alkyl, alkenyl and alkynyl radicals of Y may be substituted by halogen, cyano, nitro, C₁-C₂-alkoxy or C₁-C₄-alkoxycarbonyl; and wherein the pyrimidine radical may also carry a radical different from hydrogen in the 2-position and wherein the phenyl ring in the 5-position of the pyrimidine ring may be unsubstituted or carry 1, 2, 3, 4 or 5 radicals L which are different from hydrogen, and the pharmaceutically acceptable salts substituted 5-phenyl pyrimidines for use in therapy, in particular in therapy or treatment of cancerous diseases.

The present invention relates to substituted 5-phenyl pyrimidines of the formula I,

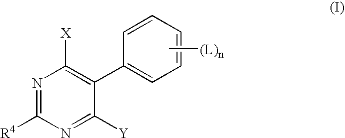

wherein
X denotes a group of the formula NR¹R², OR^{1a} or SR^{1a}, in which
R¹, R², independently of each other, denote hydrogen, C₁-C₁₀-alkyl, C₂-C₆-alkenyl, C₂-C₆-alkynyl, C₁-C₁₀-haloalkyl, C₃-C₈-cycloalkyl, C₃-C₈-halocycloalkyl, phenyl, or 5- or 6-membered heteroaryl or 5- or 6-membered heterocyclyl, containing 1, 2, 3 or 4 nitrogen atoms or 1, 2 or 3 nitrogen atoms and one sulfur or oxygen atom as ring members, which radicals may be unsubstituted or may carry 1, 2, 3 or 4 radicals R^{a1}; or
   the radical NR¹R² may also form a 5- or 6-membered optionally substituted heterocyclic ring, containing 1, 2, 3 or 4 nitrogen atoms or 1, 2 or 3 nitrogen atoms and one sulfur or oxygen atom as ring members, which are non-adjacent to the nitrogen of NR¹R², in which two adjacent C atoms or one N atom and one adjacent C atom can be linked by a C₁-C₄-alkylene chain and wherein the heterocyclic ring may be unsubstituted or may carry 1, 2, 3 or 4 radicals R^{a1}; wherein
   R^{a1} is halogen, oxo, nitro, cyano, hydroxy, C₁-C₆-alkyl, C₃-C₆-cycloalkyl, C₃-C₆-cycloalkenyl, C₁-C₆-haloalkyl, C₁-C₆-alkoxy, C₁-C₆-alkylthio, —C(═O)-A, —C(═O)—O-A, —C(═O)—N(A′)A, C(A′)(═N-OA), N(A′)A, N(A′)-C(═O)-A, N(A″)-C(═O)—N(A′)A, S(═O)ₘ-A, S(═O)ₘ—O-A, S(═O)ₘ—N(A′)A, phenyl or 5- or 6-membered heteroaryl, containing 1, 2, 3 or 4 nitrogen atoms as ring members or 1, 2 or 3 nitrogen atoms and one sulfur or oxygen atom as ring members, where the phenyl and the hetaryl moiety may carry one to three radicals selected from the group consisting of halogen, C₁-C₆-alkyl, C₂-C₆-alkenyl, C₂-C₆-alkynyl, C₃-C₆-cycloalkyl, C₁-C₈-halogenalkyl, C₁-C₆-alkoxy, cyano, nitro, —C(═O)-A, —C(═O)—O-A, —C(═O)—N(A′)A, C(A′)(═N-OA) or N(A′)A,
      wherein m is 0, 1 or 2;
      A, A′ and A″ independently of each other are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by nitro, cyanato, cyano or C₁-C₄-alkoxy; or A and A′ together with the atoms to which they are attached are a five- or six-membered saturated, partially unsaturated or aromatic heterocycle which contains one to four heteroatoms from the group consisting of O, N and S;
   R^{1a} has one of the meanings given for R¹ except for hydrogen;
   Y is a radical selected from the group consisting of halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the alkyl, alkenyl and alkynyl radicals of Y may be substituted by halogen, cyano, nitro, C₁-C₂-alkoxy or C₁-C₄-alkoxycarbonyl;
   R⁴ is a radical different from hydrogen, which comprises from 1 to 15 atoms that are different from hydrogen and which are selected from carbon, halogen, nitrogen, oxygen and sulfur, the number of carbon atoms being from 0 to 10, the number of halogen atoms being from 0 to 5 and the number of heteroatoms that are different from halogen being from 1 to 4:
   L is a radical which comprises from 1 to 10 atoms that are different from hydrogen and which are selected from carbon, halogen, nitrogen, oxygen and sulfur, the number of carbon atoms being from 0 to 10, the number of halogen atoms being from 0 to 5 and the number of heteroatoms that are different from halogen being from 0 to 4;
   n is 0, 1, 2, 3, 4 or 5;
      and the pharmaceutically acceptable salts of the substituted 5-phenyl pyrimidines I for use in therapy, in particular in therapy or treatment of cancerous diseases.

The invention also relates to pharmaceutical compositions comprising a 5-phenyl pyrimidine of the formula I as herein defined or a pharmaceutically acceptable salt thereof and a pharmaceutically acceptable carrier. Moreover the invention relates to the use of a 5-phenyl pyrimidine of the formula I as herein defined and of their pharmaceutically acceptable salts in the manufacture of a medicament for treatment of cancer and to a method for cancer treatment, which comprises administering to the subject in need thereof an effective amount of a 5-phenyl pyrimidine of the formula I as herein defined or of their pharmaceutically acceptable salts.

Despite dramatic advances in research and novel treatment options, cancer is still one of the leading cause of death. Amongst the different types of cancer such as lung, breast, prostate and colon cancer as well as colon lymphomas, are most frequently diagnosed and ovarian cancer is the 2^{nd} most common reproductive cancer after breast cancer in women. A large number of cytotoxic compounds are known to effectively inhibit the growth of tumor cells, including taxoides like paclitaxel (Taxole), docetaxel (Taxotere), the vinka alkaloids vinorelbine, vinblastine, vindesine and vincristine. However, these compounds are natural products having a complex structure and thus are difficult to produce.

It is, therefore, an object of the present invention to provide compounds which effectively control or inhibit growth and/or progeny of tumor cells and thus are useful in the treatment of cancer. It is highly desirable that these compounds can be synthesized from simple starting compounds according to standard methods of organic chemistry.

We have found that these and further objects are achieved by the substituted 5-phenyl pyrimidines I defined at the outset. Furthermore, we have found a method for treating cancer, which comprises administering to the subject in need thereof an effective amount of a 5-phenyl pyrimidine I as herein defined or of their pharmaceutically acceptable salts.

Substituted 5-phenyl pyrimidines I have been occasionally described in the literature, e.g. in WO 02/074753, WO 03/070721, WO 03/043993 and WO 2004/103978. The compounds disclosed in these documents are active against various phytopathogenic fungi. However, these documents do not describe or suggest that these compounds may be effective in the treatment of diseases or even in the treatment of cancer.

Substituted 5-phenyl pyrimidines I can be prepared by the methods disclosed in WO 02/074753, WO 03/070721, WO 03/043993, WO 2004/103978, PCT/EP04/07258 and DE 102004034197.4 and in the literature cited therein as well as by standard methods of organic chemistry.

It is likewise possible to use physiologically tolerated salts of the 5-phenyl pyrimidines I, especially acid addition salts with physiologically tolerated acids. Examples of suitable physiologically tolerated organic and inorganic acids are hydrochloric acid, hydrobromic acid, phosphoric acid, nitric acid, sulfuric acid, organic sulfonic acids having from 1 to 12 carbon atoms, e.g. C₁-C₄-alkylsulfonic acids such as methanesulfonic acid, cycloaliphatic sulfonic acids such as S-(+)-10-camphorsulfonic acids and aromatic sulfonic acids such as benzenesulfonic acid and toluenesulfonic acid, di- and tricarboxylic acids and hydroxycarboxylic acids having from 2 to 10 carbon atoms such as oxalic acid, malonic acid, maleic acid, fumaric acid, mucic acid, lactic acid, tartaric acid, citric acid, glycolic acid and adipic acid, as well as cis- and trans-cinnamic acid, furoic acid and benzoic acid. Other utilizable acids are described in Fortschritte der Arzneimittelforschung [Advances in Drug Research], Volume 10, pages 224 ff., Birkhäuser Verlag, Basel and Stuttgart, 1966. The physiologically tolérated salts of 5-phenyl pyrimidines I may be present as the mono-, bis-, tris- and tetrakis-salts, that is, they may contain 1, 2, 3 or 4 of the aforementioned acid molecules per molecule of formula I. The acid molecules may be present in their acidic form or as an anion. The acid addition salts are prepared in a customary manner by mixing the free base of a 5-phenyl pyrimidine I with a corresponding acid, where appropriate in solution in water or an organic solvent as for example a lower alcohol such as methanol, ethanol, n-propanol or isopropanol, an ether such as methyl tert-butyl ether or diisopropyl ether, a ketone such as acetone or methyl ethyl ketone, or an ester such as ethyl acetate. Solvents, wherein the acid addition salt of I is insoluble (anti-solvents), might be added to precipitate the salt. Suitable anti-solvents comprise C₁-C₄-alkylesters of C₁-C₄-aliphatic acids such as ethyl acetate, aliphatic and cycloaliphatic hydrocarbons such as hexane, cyclohexane, heptane, etc., di-C₁-C₄-alkylethers such as methyl tert-butyl ether or diisopropyl ether.

In the symbol definitions given in formula I above, collective terms were used which generally represent the following substituents:
halogen: fluorine, chlorine, bromine or iodine;
alkyl and the alkyl moieties of alkoxy, alkylthio, alkoxycarbonyl, alkylamino, di(alkyl)amino, alkylaminocarbonyl, di(alkyl)amincarbonyl, alkylcarbonylamino, alkylsulfinyl, alkylsulfonyl, alkylaminosulfonyl or di(alkyl)aminosulfonyl: saturated, straight-chain or branched hydrocarbon radicals having 1 to 10, preferably 1 to 6 carbon atoms, especially 1 to 4 carbon atoms, such as methyl, ethyl, propyl, 1-methylethyl, butyl, 1-methylpropyl, 2-methylpropyl, 1,1-dimethylethyl, or pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
alkenyl and the alkenyl moieties of alkenyloxy: unsaturated, straight-chain or branched hydrocarbon radicals having 2 to 6, preferably 2 to 4 carbon atoms, and a double bond in any position, especially C₃-C₄-alkenyl, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl and 2-methyl-2-propenyl;
alkynyl: straight-chain or branched hydrocarbon radicals having 2 to 6, preferably 2 to 4 carbon atoms, and a triple bond in any position, especially C₃-C₄-alkynyl, for example ethynyl, 1-propynyl, 2-propynyl, 1-butynyl, 2-butynyl, 3-butynyl and 1-methyl-2-propynyl;
cycloalkyl: mono- or bicyclic hydrocarbon radicals having 3 to 10 carbon atoms; monocyclic groups having 3 to 8, especially 3 to 6 ring members, for example C₃-C₈-cycloalkyl such as cyclopropyl, cyclobutyl, cyclopentyl, cyclohexyl, cycloheptyl and cyclooctyl;
haloalkyl and the haloalkyl moieties of haloalkoxy: straight-chain or branched alkyl groups having 1 to 10 carbon atoms, preferably 1 to 6 carbon atoms, especially 1 to 4 carbon atoms (as mentioned above), where the hydrogen atoms in these groups may be partially or fully replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl; similar considerations apply to other halogenated groups such as haloalkenyl and haloalkynyl where the hydrogen atoms of the alkenyl and alkynyl groups may be partially or fully replaced by halogen atoms as mentioned above;
oxy-alkyleneoxy: divalent straight-chain hydrocarbon radicals having 1 to 3 carbon atoms, e.g. OCH₂CH₂O or OCH₂CH₂CH₂O;
5- or 6-membered heterocycle: homo- or bicyclic hydrocarbon radicals containing one to four heteroatoms selected from the group consisting of a nitrogen atom, an oxygen atom and a sulfur atom; unsaturated (heterocyclyl) includes partially unsaturated, e.g. mono-unsaturated, and aromatic (heteroaryl); said heterocycles in particular include:
5-membered heteroaryl, containing one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,3-triazol-?-yl, 1,2,4-triazol-3-yl, tetrazolyl, 1,3,4-oxadiazol-2-yl, 1,3,4-thiadiazol-2-yl and 1,3,4-triazol-2-yl;
6-membered heteroaryl, containing one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,3-triazinyl, 1,3,5-triazin-2-yl and 1,2,4-triazin-3-yl.
5- and 6-membered heterocyclyl, containing one to four nitrogen atoms or one to three nitrogen atoms and one sulfur or oxygen atom: 3-pyrazolidinyl, 4-pyrazolidinyl, 5-pyrazolidinyl, 2-pyrrolodin-2-yl, 2-pyrrolodin-3-yl, 3-pyrrolodin-2-yl, 3-pyrrolodin-3-yl, 1-piperidinyl, 2-piperidinyl, 3-piperidinyl, 4-piperidinyl, pyridin(1,2-dihydro)-2-on-1-yl, 2-piperazinyl, 1-pyrimidinyl, 2-pyrimidinyl, morpholin-4-yl, thiomorpholin-4-yl.

With regard to their activity to inhibit growth and progeny of tumor cells preference is given to 5-phenyl pyrimidines I, wherein X is a radical NR¹R² in which R¹ is not hydrogen. Particularly preferred are 5-phenyl pyrimidines I, wherein X is a radical NR¹R² in which R² is hydrogen. Very particular preference is given to compounds I in which R¹ is not hydrogen and R² is hydrogen. Preference is likewise given to 5-phenyl pyrimidines I, wherein X is a radical NR¹R² in which R² is methyl or ethyl.

Particular preference is given 5-phenyl pyrimidines I, wherein X is a radical NR¹R² in which R¹ is C₁-C₆-alkyl, C₂-C₆-alkenyl or C₁-C₈-haloalkyl.

Preference is likewise given 5-phenyl pyrimidines I, wherein X is a radical NR¹R² in which R¹ is a group B:

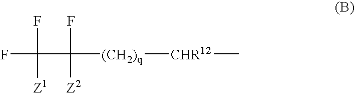

in which
Z¹ is hydrogen, fluorine or C₁-C₆-fluoroalkyl,
Z² is hydrogen or fluorine, or
Z¹ and Z² together form a double bond;
q is 0 or 1; and
R¹² is hydrogen or methyl.

Moreover, preference is given to 5-phenyl pyrimidines I, wherein X is a radical NR¹R² in which R¹ is C₃-C₆-cycloalkyl which may be substituted by C₁-C₄-alkyl.

If R¹ and/or R² contain haloalkyl or haloalkenyl groups having a center of chirality, the (S)-isomers are preferred for these groups. In the case of halogen-free alkyl or alkenyl groups having a center of chirality in R¹ or R², preference is given to the (R) configured isomers.

Preference is furthermore given to 5-phenyl pyrimidines I, wherein X is a radical NR¹R² in which R¹ and R² together with the nitrogen atom to which they are attached form a piperidinyl, morpholinyl or thiomorpholinyl ring, in particular a piperidinyl ring which is optionally substituted by one to three groups selected from halogen, C₁-C₄-alkyl or C₁-C₄-haloalkyl. Amongst these preference is given to compounds I in which R¹ and R² together with the nitrogen atom to which they are attached form a 4-methylpiperidine ring.

Preference is also given to 5-phenyl pyrimidines I, wherein the radical NR¹R² forms a pyrazole ring which is optionally substituted by one or two groups selected from halogen, C₁-C₄-alkyl or C₁-C₄-haloalkyl, in particular by 2-methyl or 3-methyl.

Preferred radicals X of the formula NR¹R² include:

NH—C₂H₅, NH(CH(CH₃)₂), NH—CH₂CH₂CH₃, NH(CH(CH₃)(C₂H₅), (S)—NHCH(CH₃)(C₂H₅), NH—CH(CH₃)(CH₂CH₂CH₃), (R)—NHCH(CH₃)(C(CH₃)₃), NH—CH(CH₃)CH(CH₃)₂, (R)—NHCH(CH₃)(CH(CH₃)₂), (S)—NHCH(CH₃)(CH(CH₃)₂), NH(cyclopentyl), NHCH₂CF₃, NHCH(CH₃)(CF₃), (R)—NHCH(CH₃)(CF₃), (S)—NHCH(CH₃)(CF₃), NH—CH(CH₃)CH₂OCH₃, NH—CH(CH₃)CH₂OH, NH—CH₂C(CH₃)═CH₂, N(CH₂CH₃)₂, N(CH₃)(CH₂CH═CH₂), N(CH₃)—CH₂CH₂CH═CH₂, N(CH₂CH═CH₂)₂, piperidin-1-yl, 2-methyl-piperidin-1-yl, 3-methyl-piperidin-1-yl, 4-methyl-piperidin-1-yl, 3,6-dihydro-2H-pyridin-1-yl, 2-methyl-pyrrolidin-1-yl, (S)—NHCH(CH₃)(C(CH₃)₃), —NH-n-butyl, —NH-tert-butyl, —NH-(sec-pentyl), —NH-2-methyl-cyclopentyl, 2-methyl-oxiranyl-methyl-amino, —N(ethyl)(isopropyl), —N(ethyl)(sec-butyl), —N(sec-butyl)₂, NHCH(CH₃)-isobutyl NH-benzyl, —NHCH(CH₃)CH₂—CH(CH₃)₂, —NH—CH(CH₃)CH₂—C(O)—OH, N(CH₂CH₃)CH₂C(CH₃)═CH₂, —N(n-Pr)(CH₂CH═CH₂), —NH—CH₂CH₂—CH₂—OH, —N(CH₃)(CH₂CH₂OH), —N(benzyl)(CH₂CH₂OH), —N(CH₂CH₂OH)(CH₂CH═CH₂)—N(CH₂CH₂OSiMe₃)(CH₂CH═CH₂), —N(CN)(CH₂CH═CH₂), —NH—CH(CH₃)CH₂—OCH₃, —NH—CH(CH₃)CH₂—C(O)—OCH₃, 2-butoxycarbonyl-pyrrolidin-1-yl, 2,5-dimethyl-pyrrolidin-1-yl, 2,6-dimethyl-morpholin-4-yl and 1,1-dioxo-thiomorpholin-4-yl.

Amongst 5-phenyl pyrimidines I, wherein X is a radical OR^{1a} or SR^{1a}, preference is given to those wherein X is OR^{1a}. The radical R^{1a} is preferably selected from C₁-C₆-alkyl, C₁-C₆-haloalkyl, C₂-C₆-alkenyl, C₂-C₆-alkinyl or C₃-C₆-cycloalkyl. In particular R^{1a} is selected from C₁-C₆-alkyl, C₂-C₆-alkenyl or C₁-C₆-haloalkyl which are branched in α-position. Likewise preferred are compounds I wherein R^{1a} is C₁-C₄-haloalkyl. Amongst these 5-phenyl pyrimidines I are especially preferred, wherein R^{1a} is ethyl, propyl, i-propyl, 1,2-dimethylpropyl, 1,2,2-trimethylpropyl, 1-methyl-2,2,2-trifluoroethyl or 2,2,2-trifluoroethyl.

Preference is given to 5-phenyl pyrimidines I, wherein Y is halogen, C₁-C₄-alkyl, cyano or C₁-C₄-alkoxy, such as chlorine, bromine, methyl, cyano, methoxy or ethoxy, especially chlorine, bromine or methyl, in particular chlorine.

The phenyl ring in the 5-phenyl pyrimidines I may be unsubstituted or preferably carries 1, 2, 3, 4 or 5, in particular 1, 2 or 3 substituents L which are different from hydrogen. Suitable radicals L usually comprises from 1 to 10 atoms that are different from hydrogen and which are selected from carbon, halogen, nitrogen, oxygen and sulfur, the number of carbon atoms are usually from 0 to 10, the number of halogen atoms are usually from 0 to 5 and the number of heteroatoms that are different from halogen are generally being from 0 to 4. Examples of suitable radicals L comprise:

halogen, cyano, cyanato (OCN), C₁-C₈-alkyl, C₂-C₁₀-alkenyl, C₂-C₁₀-alkynyl, C₁-C₆-alkoxy, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(═O)ₚ-A¹, S(═O)ₚ—O-A¹ or S(═O)ₚ—N(A²)A¹, wherein
p is 0, 1 or 2;
A¹, A², A³ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by cyano or C₁-C₄-alkoxy; or A¹ and A² together with the atoms to which they are attached are a five- or six-membered saturated, partially unsaturated or aromatic heterocycle which contains one to four heteroatoms from the group consisting of O, N and S;
where the aliphatic, alicyclic or aromatic groups of the radical definitions of L or A¹, A² or A³, respectively, for their part may be partially or fully halogenated or may carry one to four groups R^{u}:
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(═O)ₚ-A¹, S(═O)ₚ—O-A¹ or S(═O)ₚ—N(A²)A¹, where p, A¹, A², A³ are as defined above and where the aliphatic, alicyclic or aromatic groups for their part may be partially or fully halogenated or may carry one to three groups R^{ua}, R^{ub} having the same meaning as R^{u}.

In particular L is selected from the group of the radicals L^{a}, L^{b}, L^{c}, L^{d} and L^{e} as described hereinafter.

Preferably the radicals L are selected from the group consisting of halogen, cyano, nitro, C₁-C₆-alkyl, C₁-C₆-haloalkyl, C₁-C₄-alkoxy, C₁-C₄-alkylthio, C₁-C₄-alkylsulfonyl, CO—NH₂, alkylaminocarbonyl, di-C₁-C₄-alkylaminocarbonyl, C₁-C₄-alkylcarbonylamino, N—C₁-C₄-alkylcarbonyl-N—C₁-C₄-alkylamino and C₁-C₄-alkoxycarbonyl, in particular fluorine, chlorine, bromine, cyano, C₁-C₄-alkyl, C₁-C₄-haloalkyl, C₁-C₄-alkoxy or C₁-C₄-alkoxycarbonyl, especially preferably fluorine, chlorine, C₁-C₂-alkyl, such as methyl or ethyl, C₁-C₂-fluoroalkyl, such as trifluoromethyl, C₁-C₂-alkoxy, such as methoxy, or C₁-C₂-alkoxycarbonyl, such as methoxycarbonyl, SCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃

More preferably the radicals L are selected from the group consisting of halogen, cyano, nitro, C₁-C₆-alkyl, C₁-C₆-haloalkyl, C₁-C₄-alkoxy and C₁-C₄-alkoxycarbonyl, in particular fluorine, chlorine, bromine, cyano, C₁-C₄-alkyl, C₁-C₄-haloalkyl, C₁-C₄-alkoxy or C₁-C₄-alkoxycarbonyl, especially preferably fluorine, chlorine, C₁-C₂-alkyl, such as methyl or ethyl, C₁-C₂-fluoroalkyl, such as trifluoromethyl, C₁-C₂-alkoxy, such as methoxy, or C₁-C₂-alkoxycarbonyl, such as methoxycarbonyl.

Preference is given to 5-phenyl pyrimidines I, wherein one or two radical(s) L is (are) attached to one (or two) of the ortho-position(s) of the phenyl ring.

In a particular preferred embodiment of the invention the phenyl ring of the 5-phenyl pyrimidines I is of the formula C

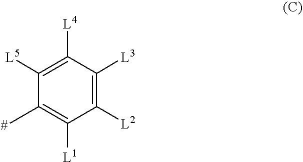

in which # is the point of attachment to the pyrimidine ring and
L¹ is hydrogen, fluorine, chlorine, CH₃ or CF₃;
L², L⁴ independently of one another are hydrogen or fluorine, in particular hydrogen;
L³ is hydrogen, fluorine, chlorine, cyano, CH₃, OCH₃ or COOCH₃; and
L⁵ is hydrogen, fluorine or CH₃,
where at least one of the radicals L¹ to L⁵ and in particular 1, 2 or 3 of the radicals L¹ to L⁵ are different from hydrogen.

The substituted 5-phenyl pyrimidines also carry a radical R⁴ in the 2-position, which is different from hydrogen. This radical R⁴ comprises from 1 to 15, in particular 2 to 15 atoms that are different from hydrogen and which are selected from carbon, halogen, nitrogen, oxygen and sulfur, the number of carbon atoms are usually from 0 to 10, the number of halogen atoms are usually from 0 to 5 and the number of heteroatoms that are different from halogen are generally being from 1 to 4. Preferred substituents in the 2-position are the radicals R^{4a}, R^{4b}, R^{4c} and R^{4d} as described hereinafter.

In a first embodiment of the invention the substituted 5-phenylpyrimidine compounds I carry a radical R^{4a} in the 2-position of the pyrimidine ring, wherein
R^{4a} denotes halogen, cyano, hydroxy, mercapto, N₃, C₁-C₆-alkyl, C₂-C₈-alkenyl, C₂-C₈-alkinyl, C₁-C₆-haloalkyl, C₁-C₆-alkoxy, C₃-C₈-alkenyloxy, C₃-C₈-alkinyloxy, C₁-C₆-haloalkoxy, C₁-C₆-alkylthio, C₃-C₈-alkenylthio, C₃-C₈-alkinylthio, C₁-C₆-haloalkylthio, or a radical of the formulae —ON═CR^{a}R^{b}, —CR^{c}═NOR^{a}, —NR^{c}N═CR^{a}R^{b}, NR^{a}R^{b}, —NR^{c}NR^{a}R^{b}NOR^{a}; —NR^{c}C(═NR^{d})—NR^{a}R^{b}, —NR^{c}C(═O)—NR^{a}R^{b}, —NR^{a}C(═O)R^{c}C, —NR^{a}C(═NOR^{c})—R^{d}, —O(C═O)R^{c}, —C(═O)—OR^{a}, —C(═O)—NR^{a}R^{b}, —C(═NOR^{c})—NR^{a}R^{b}, —CR^{c}(═NNR^{a}R^{b}), wherein
   R^{a}, R^{b}, R^{c}, R^{d} independently of each other denote hydrogen, C₁-C₆-alkyl, C₂-C₈-alkenyl, C₂-C₈-alkinyl, C₁-C₆-haloalkyl, C₁-C₆-alkoxy, C₁-C₆-haloalkoxy, R^{a} may also be C₁-C₆-alkylcarbonyl, or R^{a} and R^{b} together form a C₂-C₄-alkylene group which may be interrupted by an oxygen atom and/or comprise a double bond or R^{a} and R^{c} together form a C₂-C₄-alkylene group which may be interrupted by an oxygen atom and/or comprise a double bond;
   a cyclic radical selected from C₃-C₁₀-Cycloalkyl, phenyl and five- to ten-membered saturated, partially unsaturated or aromatic mono- or bicyclic heterocycles comprising 1, 2, 3 or 4 heteroatoms selected from the group consisting of O, N or S, it being possible for C₁-C₆-alkyl and for the cyclic radical to be partially or fully halogenated or to be substituted by 1, 2 or 3 identical or different radicals R^{x}:
   R^{x} denotes cyano, nitro, amino, aminocarbonyl, aminothiocarbonyl, hydroxy, C₁-C₆-alkyl, C₁-C₆-haloalkyl, C₁-C₆-alkylcarbonyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heteroaryl, 5- or 6-membered heterocyclyl or 5- or 6-membered heteroaryloxy, C(═NOR^{α})—OR^{β} or OC(R^{α})₂—C(R^{β})═NOR^{β},
      wherein the cyclic radicals R^{x} may be unsubstituted or substituted by 1, 2 or 3 radicals R^{y}:
      R^{y} cyano, nitro, halogen, hydroxy, amino, aminocarbonyl, aminothiocarbonyl, C₁-C₆-alkyl, C₁-C₆-haloalkyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heteroaryl, 5- or 6-membered heterocyclyl or 5- or 6-membered heteroaryloxy, or C(═NOR^{α})—OR^{β}; and
      R^{α}, R^{β} denote hydrogen or C₁-C₆-alkyl.

Preferably R^{4a} is selected from cyano, N₃, C₂-C₈-alkinyl, C₁-C₆-haloalkyl, C₃-C₈-alkenyloxy, C₃-C₈-alkinyloxy, C₁-C₆-haloalkoxy, C₃-C₈-alkenylthio, C₃-C₈-alkinylthio, C₁-C₆-haloalkylthio, or a radical of the formulae —ON═CR^{a}R^{b}, —CR^{c}═NOR^{a}, —NR^{c}N═CR^{a}R^{b}, —NR^{c}NR^{a}R^{b}, —NOR^{a}; —NR^{c}C(═NR^{d})—NR^{a}R^{b}, —NR^{c}C(═O)—NR^{a}R^{b}, —NR^{a}C(═O)R^{c}, —NR^{a}C(═NOR^{c})—R^{d}, —O(C═O)R^{c}, —C(═O)—OR^{a}, —C(═O)—NR^{a}R^{b}, —C(═NOR^{c})—NR^{a}R^{b}, —CR^{c}(═NNR^{a}R^{b}), wherein

R^{a}, R^{b}, R^{c}, R^{d} independently of each other denote hydrogen, C₁-C₆-alkyl, C₂-C₈-alkenyl, C₂-C₈-alkinyl, C₁-C₆-haloalkyl, C₁-C₆-alkoxy, C₁-C₆-haloalkoxy, R^{a} may also be C₁-C₆-alkylcarbonyl, or R^{a} and R^{b} together form a C₂-C₄-alkylene group which may be interrupted by an oxygen atom and/or comprise a double bond or R^{a} and R^{c} together form a C₂-C₄-alkylene group which may be interrupted by an oxygen atom and/or comprise a double bond;

More preferably R^{4a} is selected from halogen, cyano or a radical of the formulae —ON═CR^{a}R^{b}, CR^{c}═NOR^{a}, —NR^{c}N═CR^{a}R^{b}, —NR^{c}NR^{a}R^{b}, —NR^{c}C(═O)NR^{a}R^{b}NR^{a}C(═O)R^{c}, —NR^{a}C(═NOR^{c})—R^{d}, —C(═O)—NR^{a}R^{b}, —C(═NOR^{c})—NR^{a}R^{b}, —CR^{c}(═NNR^{a}R^{b}), wherein R^{a}, R^{b}, R^{c} and R^{d} are as defined above.

In particular R^{a} is H or C₁-C₆-alkyl, R^{b} is H or C₁-C₆-alkyl, R^{c} is H, C₁-C₆-alkyl or C₁-C₄-haloalkyl and R^{d} is H or C₁-C₆-alkyl, or R^{a} and R^{b} or R^{a} and R^{c} together form a C₂-C₄-alkylene group which may comprise a double bond.

Examples of preferred radicals R^{4a} include:

2-oxo-pyrrolidin-1-yl, —C(CH₃)═NOH, —C(NH₂)═NOH, —C(NH₂)═NOCH₃, —C(NH₂)═NOC₂H₅, —C(NH₂)═NOCHF₂, —C(O)NH₂, —C(O)NH(CH₃), —C(O)NHC(O)CH₃, —CN, —N(CH₃)NH₂, —NHN═CH(CH(CH₃)C(═O)OC₂H₅) and —ON═C(CH₃)₂.

Amongst the 5-phenyl pyrimidines I, which carry a radical R^{4a} in the 2-position of the pyrimidine moiety, compounds formula Ia

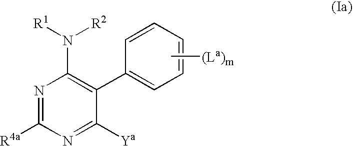

are preferred, in which R¹, R² and R^{4a} have the meanings given above,
m is 1, 2, 3, 4 or 5, in particular 1, 2 or 3;
Y^{a} denotes halogen, cyano, C₁-C₆-alkyl, C₁-C₆-haloalkyl, C₁-C₆-alkoxy, C₁-C₄-haloalkoxy or C₃-C₆-alkenyloxy; in particular C₁-C₄-alkyl, cyano or C₁-C₄-alkoxy, such as chlorine, bromine, methyl, cyano, methoxy or ethoxy, especially chlorine, bromine or methyl, most preferably chlorine;
L^{a} denotes, independently of each other, halogen, C₁-C₆-alkyl, C₁-C₆-alkoxy and C₁-C₆-haloalkyl. In particular the phenyl ring of the compounds Ia is of the formula C as defined above.

In a second embodiment of the invention the substituted 5-phenylpyrimidine compounds I carry a radical R^{4b} in the 2-position of the pyrimidine ring, wherein R^{4b} denotes a five- to ten-membered saturated, partially unsaturated or aromatic mono- or bicyclic heterocycle comprising one to four hetero atoms selected from the group consisting of O, N or S, it being possible for R^{4b} to be substituted by one to three identical or different groups R⁴⁴, wherein
R⁴⁴ is halogen, hydroxyl, cyano, oxo, nitro, amino, mercapto, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, carboxyl, C₁-C₆-alkoxycarbonyl, carbamoyl, C₁-C₆-alkylaminocarbonyl, C₁-C₆-alkyl-C₁-C₆-alkylamincarbonyl, morpholinocarbonyl, pyrrolidinocarbonyl, C₁-C₆-alkylcarbonylamino, C₁-C₆-alkylamino, di(C₁-C₆-alkyl)amino, C₁-C₆-alkylthio, C₁-C₆-alkylsulfinyl, C₁-C₆-alkylsulfonyl, hydroxysulfonyl, aminosulfonyl, C₁-C₆-alkylaminosulfonyl, di(C₁-C₆-alkyl)aminosulfonyl, phenyl, 5- or 6-membered heteroaryl comprising one to four hetero atoms selected from the group consisting of O, N or S it being possible for the alkyl, phenyl, heteroaryl, cycloalkyl and alkoxy groups in the radicals R⁴⁴ to be partially or fully halogenated or to be substituted by 1, 2 or 3 identical or different radicals R^{x} as defined above.

Preferably the radical R^{4b} is selected from an aromatic heterocyclic radical which comprises 1, 2 or 3 nitrogen atoms as ring members or 1 or 2 nitrogen atoms and 1 oxygen atom or 1 sulfur atom as ring members, in particular pyrazol, in particular pyrazol-1-yl, thiazol, in particular thiazol-2-yl or thiazol-4-yl, 1,2,3-triazol, in particular 1,2,3-triazol-1-yl or 1,2,3-triazol-2-yl, 1,2,4-triazol, in particular 1,2,4-triazol-1-yl, pyridyl, in particular pyridin-2-yl, pyrazin, in particular pyrazin-2-yl, and pyridazin, in particular pyridazin-3-yl. The aforementioned aromatic heterocyclic radicals may carry 1, 2 or 3 identical or different groups R⁴⁴ as defined above, in particular a radical R⁴⁴ which is selected from halogen, cyano, nitro, amino, C₁-C₄-alkyl, C₁-C₄-alkoxy, C₁-C₄-alkoxycarbonyl, C₁-C₄-alkylcarbonyloxy, C₁-C₄-haloalkyl, C₁₋₄-haloalkoxy, C₁-C₄-alkylthio, C₁-C₄-alkylsulfonyl, —S—CH₂—C₆H₅ (benzylthio), phenyl or furyl.

Examples of preferred radicals R^{4b} include:

pyrazol-1-yl, 3-amino-pyrazol-1-yl, 3-(i-propyl)pyrazol-1-yl, 3-bromo-pyrazol-1-yl, 3-CH₃-pyrazol-1-yl, 3-CF₃-pyrazol-1-yl, 3-phenylpyrazol-1-yl, 4-bromo-pyrazol-1-yl, 4-chloro-pyrazol-1-yl, 4-iodo-pyrazol-1-yl, 4-CH₃-pyrazol-1-yl, 4-cyano-pyrazol-1-yl, 5-nitropyrazol-1-yl, 3-amino-4-cyano-pyrazol-1-yl, 3-(furan-2-yl)-4-methyl-pyrazol-1-yl, 4-methyl-5-oxo-2,5-dihydro-pyrazol-1-yl, 5-chloro-4-methyl-pyrazol-1-yl, 5-ethoxycarbonyl-3-methyl-pyrazol-1-yl, 5-methoxy-4-methyl-pyrazol-1-yl, 3,5-dimethylpyrazol-1-yl, 3,5-dimethyl-4-chloropyrazol-1-yl, 1,2,3-triazol-1-yl, 1,2,3-triazol-2-yl, 1,2,4-triazol-1-yl, 3-amino-1,2,4-triazol-1-yl, 3-benzylsulfanyl-1,2,4-triazol-1-yl, 3-nitro-1,2,4-triazol-1-yl, 3,5-dimethyl-1,2,4-triazol-1-yl, thiazol-2-yl, 2-methyl-thiazol-4-yl, 4-methyl-thiazol-2-yl, 2-pyridyl, 4-CH₃-pyrid-2-yl, 6-CH₃-pyrid-2-yl, pyrazin-2-yl and pyridazin-3-yl.

Amongst the 5-phenyl pyrimidines I, which carry a radical R^{4b} in the 2-position of the pyrimidine moiety, compounds formula Ib

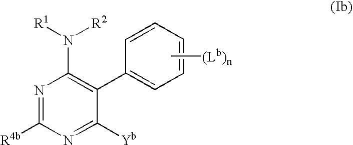

are preferred in which R¹, R² and R^{4b} are as define above,
n is 1, 2, 3, 4 or 5, in particular 1, 2, or 3;
Y^{b} denotes halogen, cyano, C₁-C₆-alkyl, C₁-C₆-haloalkyl, C₁-C₆-alkoxy, C₁-C₄-haloalkoxy or C₃-C₆-alkenyloxydenotes halogen, cyano, C₁-C₆-alkyl, C₁-C₆-haloalkyl, C₁-C₆-alkoxy, C₁-C₄-haloalkoxy or C₃-C₆-alkenyloxy; in particular C₁-C₄-alkyl, cyano or C₁-C₄-alkoxy, such as chlorine, bromine, methyl, cyano, methoxy or ethoxy, especially chlorine, bromine or methyl, most preferably chlorine;
L^{b} denotes, independently of each other, halogen, C₁-C₆-alkyl, C₁-C₆-alkoxy, C₁-C₆-haloalkyl, C₁-C₆-haloalkoxy, C₃-C₆-cycloalkoxy, C₁-C₆-alkoxycarbonyl and C₁-C₆-alkylaminocarbonyl. In particular the phenyl ring of the compounds Ib is of the formula C as defined above.

In a third embodiment of the invention the substituted 5-phenylpyrimidine compounds I carry a radical R^{4c} in the 2-position of the pyrimidine ring, wherein

R^{4c} corresponds to one of the formulae:

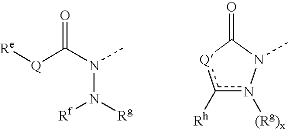

where
x is 0 or 1;
R^{e}, R^{f}, R^{g}, R^{e#} independently of one another are hydrogen, C₁-C₆-alkyl, C₂-C₈-alkenyl, C₂-C₈-alkynyl, C₃-C₆-cycloalkyl, C₄-C₆-cycloalkenyl,
R^{f}, R^{g} together with the nitrogen atom to which they are attached may have the meaning R^{e}-Z-C(R^{h})═N;
Q is oxygen or N—R^{e#};
Q′ is C(H)—R^{k}, C—R^{k}, N—N(H)—R^{e#} or N—R^{e#};
[symbol] may be a double bond or a single bond;

R^{h}, R^{k} have the same meanings as R^{e} and may additionally be halogen or cyano;

R^{h} together with the carbon to which it is attached may be a carbonyl group;
where the aliphatic, alicyclic or aromatic groups of the radical definitions of R^{e}, R^{e#}, R^{f}, R^{g}, R^{h} or R^{k} for their part may be partially or fully halogenated or may carry one to four groups R^{v}:
R^{v} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, and where two of the radicals R^{f}, R^{g}, R^{e} or R^{e#} together with the atoms to which they are attached may form a five- or six-membered saturated, partially unsaturated or aromatic heterocycle which contains one to four heteroatoms from the group consisting of O, N and S.

Preferably, the radical R^{4c} corresponds one of the following formulae:

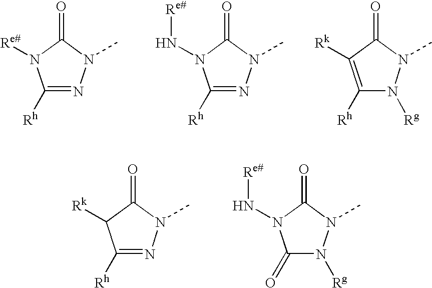

wherein R^{e#}, R^{g} and R^{h} are as defined above. In these formulae R^{e#}, R^{g} and R^{h} are preferably independently of one another hydrogen, C₁-C₆-alkyl, C₂-C₆-alkenyl, C₂-C₆-alkynyl or C₃-C₆-cycloalkyl, in particular are hydrogen, methyl or ethyl. Amongst these preference is given to radicals R^{4c} of the formulae:

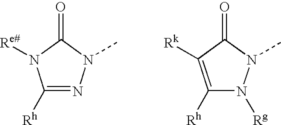

wherein R^{e#}, R^{g} and R^{h} are as defined above. Examples for these radicals include radicals of the following formulae:

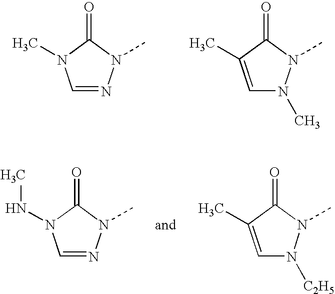

Likewise, preference is given to 5-phenyl pyrimidines I, wherein the radical R^{4c} in the 2-position is of the formula:

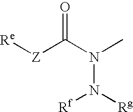

wherein Z, R^{e}, R^{f} and R^{g} are as defined above. Preferably Z is oxygen. Preferably R^{e}, R^{f} and R^{g} are independently of one another hydrogen, C₁-C₆-alkyl, C₂-C₆-alkenyl, C₂-C₆-alkynyl or C₃-C₆-cycloalkyl, in particular hydrogen, methyl or ethyl or R^{f} and R^{g} together with the nitrogen are a radical R^{e}-Z-C(R^{h})═N, wherein Z, R^{e} and R^{h} are as defined above. In particular Z is oxygen and R^{e} and R^{h} are H or C₁-C₆-alkyl. Examples of this type of radical R^{4c} include:

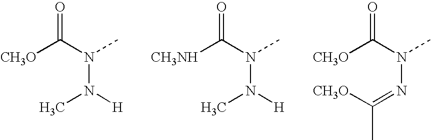

Amongst the 5-phenyl pyrimidines I, which carry a radical R^{4c} in the 2-position of the pyrimidine moiety, compounds formula Ic

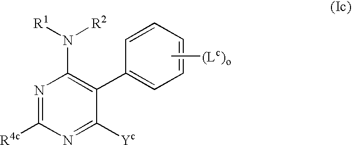

in which R¹, R² and R^{4c} have the meanings given above,
o is 1, 2, 3, 4 or 5, in particular 1, 2 or 3;
Y^{c} is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy, where the alkyl, alkenyl and alkynyl radicals of Y^{c} may be substituted by halogen, cyano, nitro, C₁-C₂-alkoxy or C₁-C₄-alkoxycarbonyl, in particular C₁-C₄-alkyl, cyano or C₁-C₄-alkoxy, such as chlorine, bromine, methyl, cyano, methoxy or ethoxy, especially chlorine, bromine or methyl, most preferably chlorine;
L^{c} is halogen, cyano, cyanato (OCN), C₁-C₈-alkyl, C₂-C₁₀-alkenyl, C₂-C₁₀-alkynyl, C₁-C₆-alkoxy, —C(═O)-A¹, —C(═O)—O-A¹, —C(═O)—N(A²)A¹, C(A²) (═N-OA¹), N(A²)A¹, N(A²)-C(═O)-A¹, N(A³)-C(═O)—N(A²)A¹, S(═O)ₚ-A¹, S(═O)ₚ—O-A¹ or S(═O)ₚ—N(A²)A¹,
   p is 0, 1 or 2;
   A¹, A², A³ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by cyano or C₁-C₄-alkoxy; or A¹ and A² together with the atoms to which they are attached are a five- or six-membered saturated, partially unsaturated or aromatic heterocycle which contains one to four heteroatoms from the group consisting of O, N and S;
   where the aliphatic, alicyclic or aromatic groups of the radical definitions of L^{c} for their part may be partially or fully halogenated or may carry one to four groups R^{u}:
   R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(═O)ₚ-A¹, S(═O)ₚ—O-A¹ or S(═O)ₚ—N(A²)A¹, where p, A¹, A², A³ are as defined above and where the aliphatic, alicyclic or aromatic groups for their part may be partially or fully halogenated or may carry one to three groups R^{ua}, R^{ub} having the same meaning as R^{u}.

Particular preference is also given to compounds Ic in which Y^{c} is C₁-C₄-alkyl which may be substituted by halogen. Moreover, particular preference is given to compounds Ic in which Y^{c} is halogen, cyano, C₁-C₄-alkyl or C₁-C₄-alkoxy. Especially preferred are compounds I in which Y^{c} is methyl, ethyl, cyano, bromine or in particular chlorine.

Moreover, particular preference is given to compounds Ic in which the index o and the substituents L^{c} are as defined below:
o is 1 to 3;
L^{c} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, —C(═O)—O-A¹, —C(═O)—N(A²)A¹, C(A³)(═N-OA¹), N(A²)A¹, N(A³)-C(═O)-A¹ or S(═O)ₘ-A¹;
   m is 0, 1 or 2;
   A¹, A², A³ independently of one another are hydrogen, C₁-C₆-alkyl, C₂-C₆-alkenyl, C₂-C₆-alkynyl, where the organic radicals may be partially or fully halogenated or may be substituted by cyano or C₁-C₄-alkoxy, or A¹ and A² together with the atoms to which they are attached are a five- or six-membered saturated heterocycle which contains one to four heteroatoms from the group consisting of O, N and S.

Especially preferred are compounds Ic, where the substituent L^{c} is as defined below:
L^{c} is halogen, cyano, C₁-C₈-alkyl, C₁-C₆-alkoxy, —C(═O)—O-A¹, —C(═O)—N(A²)A³,
   m is 0, 1 or 2;
   A¹, A², independently of one another are hydrogen, C₁-C₆-alkyl, C₂-C₆-alkenyl, C₂-C₆-alkynyl which radicals may carry a radical R^{u} as defined above.

R^{u} is preferably halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₅-C₆-cycloalkenyl, —C(═O)—O-A¹, —C(═O)—N(A²)A¹, C(A²)(═N-OA¹), where the aliphatic or alicyclic groups for their part may be partially or fully halogenated or may carry one to three groups R^{v}, R^{v} having the same meaning as R^{u}. R^{u} is in particular halogen, cyano, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₃-C₆-cycloalkyl, C₅-C₆-cycloalkenyl.

Amongst compounds Ic preference is given to compounds Ic′

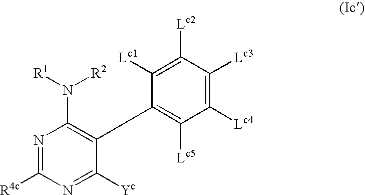

wherein R¹, R², R^{4c} and Y^{c} are as defined above and wherein
L^{c1} is fluorine, chlorine, CH₃ or CF₃;
L^{c2}, L^{c4} independently of one another are hydrogen, CH₃ or fluorine;
L^{c3} is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L^{c5} is hydrogen, fluorine, chlorine or CH₃.

In a fourth embodiment of the invention the substituted 5-phenyl pyrimidine compounds I carry a radical R^{4d} in the 2-position of the pyrimidine ring, wherein

R^{4d} corresponds to one of the formulae

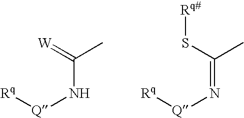

where
Q″ is a direct bond, —(C═O)—, —(C═O)—NH, —(C═O)—O—, —O—, —NR^{p}—, where the molecule moiety to the left in each case is attached to the nitrogen atom;
R^{p} is hydrogen, methyl or C₁-C₄-acyl (═C₁-C₄-alkylcarbonyl) and
R^{q} is hydrogen, methyl, benzyl, trifluoromethyl, allyl, propargyl or methoxymethyl;
R^{q#} is hydrogen, C₁-C₆-alkyl; C₂-C₆-alkynyl;
W is S or NR^{q#};
   where the aliphatic groups of the radical definitions of R^{p}, R^{q} and/or R^{q#} for their part may carry one or two groups R^{w}:
R^{w} is halogen, OR^{z}, NHR^{z}, C₁-C₆-alkyl, C₁-C₄-alkoxycarbonyl, C₁-C₄-acylamino, [1,3]dioxolane-C₁-C₄-alkyl, [1,3]dioxane-C₁-C₄-alkyl, where R^{z} is hydrogen, methyl, allyl or propargyl.

Preferred radicals R^{4d} are of the following formulae

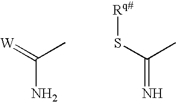

wherein W and R^{q#} are as defined above.

Finally, R^{4d} may preferably have the following meanings, which may also be understood as prodrug radical definitions (see Medicinal Research Reviews 2003, 23, 763-793, or J. of Pharmaceutical Sciences 1997, 86, 765-767):

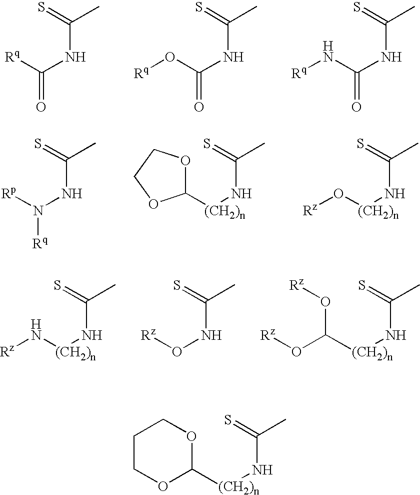

In the ten aforementioned radicals the index n in the alkenyl radicals of the above formulae is an integer from 1, 2 or 3. The substituent R^{z} is preferably hydrogen, methyl, allyl or propargyl and particularly preferably hydrogen. The substituent R^{q} is preferably hydrogen, C₁-C₆-alkyl or C₂-C₆-alkenyl and with particular preference methyl, allyl or propargyl.

Amongst the 5-phenyl pyrimidines I, which carry a radical R^{4d} in the 2-position of the pyrimidine moiety, compounds formula Id

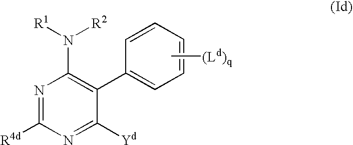

are preferred, in which R¹, R² and R^{4d} have the meanings given in claim 1,
q is 1, 2, 3, 4 or 5, in particular 1, 2 or 3;
Y^{d} is halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the alkyl, alkenyl and alkynyl radicals of Y^{d} may be substituted by halogen, cyano, nitro, C₁-C₂-alkoxy or C₁-C₄-alkoxycarbonyl. Y^{d} is in particular C₁-C₄-alkyl, cyano or C₁-C₄-alkoxy, such as chlorine, bromine, methyl, cyano, methoxy or ethoxy, especially chlorine, bromine or methyl, most preferably chlorine;
L^{d} has one of the meanings given for L^{c}.

Particular preference is also given to compounds Id in which Y^{d} is C₁-C₄-alkyl which may be substituted by halogen. Moreover, particular preference is given to compounds Ic in which Y^{d} is halogen, cyano, C₁-C₄-alkyl or C₁-C₄-alkoxy. Especially preferred are compounds I in which Y^{d} is methyl, ethyl, cyano, bromine or in particular chlorine.

Amongst compounds Id preference is given to compounds Id′

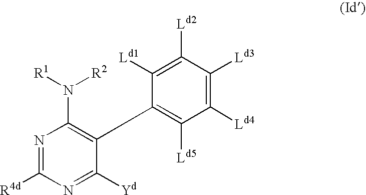

wherein R¹, R², R^{4d} and Y^{d} are as defined above and wherein
L^{d1} is fluorine, chlorine, CH₃ or CF₃;
L^{d2}, L^{d4} independently of one another are hydrogen, CH₃ or fluorine;
L^{d3} is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L^{d5} is hydrogen, fluorine, chlorine or CH₃.

In another embodiment of the invention, the substituted 5-phenyl pyrimidines I are of formula Ie

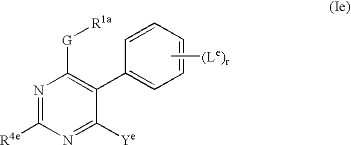

in which R^{1a} is as defined in claim 1,
r is 1, 2, 3, 4 or 5, in particular 1, 2 or 3;
Y^{e} is halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the alkyl, alkenyl and alkynyl radicals of Y^{e} may be substituted by halogen, cyano, nitro, C₁-C₂-alkoxy or C₁-C₄-alkoxycarbonyl;
G denotes O or S, in particular O;
L^{e} has one of the meanings given for L^{c}, in particular one of the preferred meanings.
R^{4e} has one of the meanings given for R^{a} or R^{4a}, in particular one of the preferred meanings.

Y^{e} is in particular halogen, C₁-C₄-alkyl, cyano or C₁-C₄-alkoxy, such as chlorine, bromine, methyl, cyano, methoxy or ethoxy, especially chlorine, bromine or methyl, most preferably chlorine.

Amongst compounds Ie preference is given to compounds Ie′

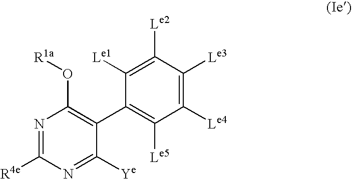

wherein R¹, R², R^{4e} and Y^{e} are as defined above and wherein
L^{e1} is fluorine, chlorine, CH₃ or CF₃;
L^{e2}, L^{e4} independently of one another are hydrogen, CH₃ or fluorine;
L^{e3} is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L^{e5} is hydrogen, fluorine, chlorine or CH₃.

The substituted 5-phenyl pyrimidines I, in particular the compounds of the formulae Ia, Ib, Ic, Id and Ie effectively inhibit growth and/or progeny of tumor cells as can be shown by standard tests on tumor cell lines such as HeLa, MCF-7 and COLO 205. In particular, 5-phenyl pyrimidines I show in general IC₅₀ values <10⁻⁶ mol/l (i.e. <1 μM), preferably IC₅₀ values <10⁻⁷ mol/l (i.e. <100 nM) for cell cycle inhibition in HeLa cells as determined by the test procedure outlined below.

Based on the results of these standard pharmacological test procedures, substituted 5-phenyl pyrimidines are useful as agents for treating, inhibiting or controlling the growth and/or progeny of cancerous tumor cells and associated diseases in a subject in need thereof. Therefore these compounds are useful in therapy of cancer in warm blooded vertebrates, i.e. mammals and birds, in particular human beings but also in other mammals of economic and/or social importance e.g. carnivores such as cats and dogs, swine (pigs, hogs and wild boars), ruminats (e.g. cattle, oxen, sheep, deer, goats, bison) and horses, or bird in particular poultry such as turkeys, chickens, ducks, geese, guinea fowl and the like.

In particular 5-phenyl pyrimidines I are useful in therapy of cancer or cancerous disease including cancer of breast, lung, colon, prostate, melanoma, epidermal, kidney bladder, mouth, larynx, esophagus, stomach, ovary, pancreas, liver, skin and brain.

The effective dosage of active ingredient employed may vary depending on the particular compound employed, the mode of administration and severity of the condition being treated. However, in general satisfactory results are obtained when the compounds of the invention are administered in amounts ranging from about 0.10 to about 100 mg/kg of body weight per day. A preferred regimen for optimum results would be from about 1 mg to about 20 mg/kg of body weight per day and such dosage units are employed that a total of from about 70 mg to about 1400 mg of the active compound for a subject of about 70 kg of body weight are administered in a 24 hour period.

The dosage regimen for treating mammals may be adjusted to provide the optimum therapeutic response. For example, several divided doses may be administered daily or the dose may be proportionally reduced as indicated by the exigencies of the therapeutic situation. A decidedly practical advantage is that these active compounds may be administered in any convenient manner such as by the oral, intravenous, intramuscular or subcutaneous routes. The active compounds may be orally administered, for example, with an inert diluent or with an assimilable edible carrier, or they may be enclosed in hard or soft shell gelatine capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 10 and 1000 mg of active compound.

The tablets, troches, pills, capsules and the like may also contain the following: a binder such as gum tragacanth, acacia, corn starch or gelatine; excipients such as dicalcium phosphate; a disintegrating agent such as corn starch, potato starch, alginic acid and the like; a lubricant such as magnesium stearate; and a sweetening agent such as sucrose, lactose, or saccharin may be added or a flavoring agent such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose, as a sweetening agent, methyl and propylparabens as preservatives, a dye and flavoring such as cherry or orange flavor. Of course, any material used in preparing any dosage unit form should be pharmaceutically pure and substantially non-toxic in the amounts used. In addition, these active compounds may be incorporated into sustained-release preparations and formulations.

These active compounds may also be administered parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth or microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be prepared against the contaminating action of microorganisms such as bacteria and fungi. The carrier can be a solvent or dispersion medium containing, for example, water, ethanol, polyol (e.g., glycerol, propylene glycol and liquid poly-ethylene glycol), suitable mixtures thereof, and vegetable oils.

The following examples 1 to 221 given in table 1 are representative compounds of this invention which are useful as anticancer agents. In table 1 the compounds are defined by formula I-A, wherein for the respective example R¹, R², R⁴, Y, (L)ₘ are given in the rows of table 1.

TABLE 1
compounds of the general formula I-A

| | |
|---|---|
| | (I-A) |
| 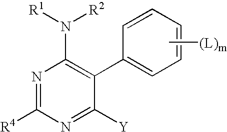 | |

| Example | R⁴ | NR¹R² | Y | (L)ₘ |
|---|---|---|---|---|
| 1 | pyrazol-1-yl | (S)-NHCH(CH₃)(CF₃) | Cl | 2,4,6-F₃ |
| 2 | 2-pyridyl | NH—CH(CH₃)₂ | Cl | 2,4,6-F₃ |
| 3 | 3,5-(CH₃)₂-4-Cl-pyrazol-1-yl | (S)-NHCH(CH₃)(CF₃) | Cl | 2,4,6-F₃ |
| 4 | 3-phenylpyrazol-1-yl | (S)-NHCH(CH₃)(CF₃) | Cl | 2,4,6-F₃ |
| 5 | 3-(i-propyl)pyrazol-1-yl | (S)-NHCH(CH₃)(CF₃) | Cl | 2,4,6-F₃ |
| 6 | 3-CF₃-pyrazol-1-yl | (S)-NHCH(CH₃)(CF₃) | Cl | 2,4,6-F₃ |
| 7 | 5-nitropyrazol-1-yl | (S)-NHCH(CH₃)(CF₃) | Cl | 2,4,6-F₃ |
| 8 | 1,2,4-triazol-1-yl | (S)-NHCH(CH₃)(CF₃) | Cl | 2,4,6-F₃ |
| 9 | —N(CH₃)NH₂ | (S)-NHCH(CH₃)(CF₃) | Cl | 2,4,6-F₃ |
| 10 | —CN | (S)-NHCH(CH₃)(CF₃) | Cl | 2,4,6-F₃ |
| 11 | 6-CH₃-pyrid-2-yl | NH—CH(CH₃)₂ | Cl | 2,4,6-F₃ |
| 12 | pyrid-2-yl | (S)-NHCH(CH₃)(CF₃) | Cl | 2,4,6-F₃ |
| 13 | 6-CH₃-pyrid-2-yl | (S)-NHCH(CH₃)(CF₃) | Cl | 2,4,6-F₃ |
| 14 | 4-CH₃-pyrid-2-yl | (S)-NHCH(CH₃)(CF₃) | Cl | 2,4,6-F₃ |
| 15 | 4-CH₃-pyrid-2-yl | NH—CH(CH₃)₂ | Cl | 2,4,6-F₃ |
| 16 | 3-CF₃-pyrazol-1-yl | NH—CH(CH₃)₂ | Cl | 2,4,6-F₃ |
| 17 | 4-Br-pyrazol-1-yl | NH—CH(CH₃)₂ | Cl | 2,4,6-F₃ |
| 18 | 3-CH₃-pyrazol-1-yl | NH—CH(CH₃)₂ | Cl | 2,4,6-F₃ |
| 19 | 4-Br-pyrazol-1-yl | NH—CH(CH₃)₂ | Cl | 2-F, 6-Cl |
| 20 | 3-CH₃-pyrazol-1-yl | NH—CH(CH₃)₂ | Cl | 2-F, 6-Cl |
| 21 | 3,5-dimethyl-pyrazol-1-yl | NH—CH(CH₃)₂ | Cl | 2,4,6-F₃ |
| 22 | 3-(i-propyl)pyrazol-1-yl | NH—CH(CH₃)₂ | Cl | 2,4,6-F₃ |
| 23 | 5-nitropyrazol-1-yl | NH—CH(CH₃)₂ | Cl | 2,4,6-F₃ |
| 24 | 4-CH₃-pyrazol-1-yl | NH—CH(CH₃)₂ | Cl | 2,4,6-F₃ |
| 25 | pyrazin-2-yl | NH—CH(CH₃)₂ | Cl | 2-F, 6-Cl |
| 26 | pyrazin-2-yl | N(CH₂CH₃)₂ | Cl | 2,4,6-F₃ |
| 27 | pyrazin-2-yl | (S)-NHCH(CH₃)(CF₃) | Cl | 2,4,6-F₃ |
| 28 | 1,2,4-triazol-1-yl | 4-methyl-piperidin-1-yl | Cl | 2,4,6-F₃ |
| 29 | 1,2,3-triazol-1-yl | 4-methyl-piperidin-1-yl | Cl | 2,4,6-F₃ |
| 30 | 3,5-dimethyl-pyrazol-1-yl | 4-methyl-piperidin-1-yl | Cl | 2,4,6-F₃ |
| 31 | 5-nitropyrazol-1-yl | 4-methyl-piperidin-1-yl | Cl | 2,4,6-F₃ |
| 32 | 3-methyl-pyrazol-1-yl | 4-methyl-piperidin-1-yl | Cl | 2,4,6-F₃ |
| 33 | 4-methyl-pyrazol-1-yl | (S)-NHCH(CH₃)(CF₃) | Cl | 2,4,6-F₃ |
| 34 | 4-iodo-pyrazol-1-yl | (S)-NHCH(CH₃)(CF₃) | Cl | 2,4,6-F₃ |
| 35 | 4-chloro-pyrazol-1-yl | (S)-NHCH(CH₃)(CF₃) | Cl | 2,4,6-F₃ |
| 36 | pyridazin-3-yl | (S)-NHCH(CH₃)CH(CH₃)₂ | Cl | 2,4,6-F₃ |
| 37 | pyrazin-2-yl | 4-methyl-piperidin-1-yl | Cl | 2,4,6-F₃ |
| 38 | 3-bromo-pyrazol-1-yl | (S)-NHCH(CH₃)(CF₃) | Cl | 2,4,6-F₃ |
| 39 | thiazol-2-yl | 4-methyl-piperidin-1-yl | Cl | 2,4,6-F₃ |
| 40 | thiazol-2-yl | NH(cyclopentyl) | Cl | 2,4,6-F₃ |
| 41 | pyrazol-1-yl | 3,6-dihydro-2H-pyridin-1-yl | Cl | 2,4,6-F₃ |
| 42 | 1,2,3-triazol-1-yl | 3-methyl-piperidin-1-yl | Cl | 2,4,6-F₃ |
| 43 | pyrazol-1-yl | 3-methyl-piperidin-1-yl | Cl | 2,4,6-F₃ |
| 44 | 1,2,4-triazol-1-yl | 3-methyl-piperidin-1-yl | Cl | 2,4,6-F₃ |
| 45 | 1,2,3-triazol-1-yl | 3,6-dihydro-2H-pyridin-1-yl | Cl | 2,4,6-F₃ |
| 46 | pyrazol-1-yl | (R)-NHCH(CH₃)(CH(CH₃)₂) | Cl | 2-F, 6-Cl |
| 47 | 1,2,4-triazol-1-yl | 4-methyl-piperidin-1-yl | Cl | 2-F, 6-Cl |
| 48 | 1,2,4-triazol-1-yl | (R)-NHCH (CH₃)(CH(CH₃)₂) | Cl | 2-F, 6-Cl |
| 49 | 1,2,3-triazol-1-yl | 4-methyl-piperidin-1-yl | Cl | 2-F, 6-Cl |
| 50 | 1,2,3-triazol-1-yl | (R)-NHCH(CH₃)(CH(CH₃)₂) | Cl | 2-F, 6-Cl |
| 51 | pyrazol-1-yl | piperidin-1-yl | Cl | 2,4,6-F₃ |
| 52 | 1,2,4-triazol-1-yl | piperidin-1-yl | Cl | 2,4,6-F₃ |
| 53 | 4-bromo-pyrazol-1-yl | piperidin-1-yl | Cl | 2,4,6-F₃ |
| 54 | 3,5-dimethyl-1,2,4-triazol-1-yl | piperidin-1-yl | Cl | 2,4,6-F₃ |
| 55 | 4-methyl-pyrazol-1-yl | piperidin-1-yl | Cl | 2,4,6-F₃ |
| 56 | 1,2,3-triazol-1-yl | piperidin-1-yl | Cl | 2,4,6-F₃ |
| 57 | 3-aminopyrazol-1-yl | NHCH(CH₃)(CF₃) | Cl | 2,4,6-F₃ |
| 58 | —C(NH₂)═NOH | 4-methyl-piperidin-1-yl | Cl | 2,4,6-F₃ |
| 59 | 3,5-dimethyl-1,2,4-triazol-1-yl | 3,6-dihydro-2H-pyridin-1-yl | Cl | 2,4,6-F₃ |
| 60 | 1,2,4-triazol-1-yl | (R)-NHCH(CH₃)(CH(CH₃)₂) | Cl | 2,4,6-F₃ |
| 61 | 2-pyridyl | 4-methyl-piperidin-1-yl | Cl | 2,6-F₂, 4-OCH₃ |
| 62 | 2-pyridyl | NH(CH(CH₃)₂) | Cl | 2,6-F₂, 4-OCH₃ |
| 63 | 2-pyridyl | NH(CH(CH₃)(C₂H₅) | Cl | 2,6-F₂, 4-OCH₃ |
| 64 | 2-pyridyl | NH(cyclopentyl) | Cl | 2,6-F₂, 4-OCH₃ |
| 65 | 2-pyridyl | (S)-NHCH(CH₃)(CH(CH₃)₂) | Cl | 2,6-F₂, 4-OCH₃ |
| 66 | pyrazol-1-yl | 4-methyl-piperidin-1-yl | Cl | 2-F, 6-Cl |
| 67 | pyrazol-1-yl | 4-methyl-piperidin-1-yl | Cl | 2,6-F₂, 4-OCH₃ |
| 68 | 1,2,4-triazol-1-yl | 4-methyl-piperidin-1-yl | Cl | 2,6-F₂, 4-OCH₃ |
| 69 | 1,2,3-triazol-1-yl | 4-methyl-piperidin-1-yl | Cl | 2,6-F₂, 4-OCH₃ |
| 70 | 2-methyl-thiazol-4-yl | (R)-NHCH(CH₃)(CH(CH₃)₂) | Cl | 2,4,6-F₃ |
| 71 | 2-methyl-thiazol-4-yl | NHCH(CH₃)(C₂H₅) | Cl | 2,4,6-F₃ |
| 72 | 2-methyl-thiazol-4-yl | NH(cyclopentyl) | Cl | 2,4,6-F₃ |
| 73 | 2-pyridyl | 4-methyl-piperidin-1-yl | Cl | 2,6-F₂, 4-OH |
| 74 | pyrazol-1-yl | 2-methyl-pyrrolidin-1-yl | Cl | 2,4,6-F₃ |
| 75 | 1,2,4-triazol-1-yl | 2-methyl-pyrrolidin-1-yl | Cl | 2,4,6-F₃ |
| 76 | 1,2,3-triazol-1-yl | 2-methyl-pyrrolidin-1-yl | Cl | 2,4,6-F₃ |
| 77 | 3,5-dimethyl-1,2,4-triazol-1-yl | 2-methyl-pyrrolidin-1-yl | Cl | 2,4,6-F₃ |
| 78 | pyridazin-3-yl | (S)-NHCH(CH₃)(CF₃) | Cl | 2,4,6-F₃ |
| 79 | pyridazin-3-yl | 4-methyl-piperidin-1-yl | Cl | 2,4,6-F₃ |
| 80 | pyridazin-3-yl | NH—CH(CH₃)CH(CH₃)₂ | Cl | 2,4,6-F₃ |
| 81 | 2-pyridyl | 4-methyl-piperidin-1-yl | Cl | 2,6-F₂ |
| 82 | 2-pyridyl | (S)-NH—CH(CH₃)CH(CH₃)₂ | Cl | 2,6-F₂ |
| 83 | 2-pyridyl | NH—CH(CH₃)₂ | Cl | 2,6-F₂ |
| 84 | 2-pyridyl | (R)-NH—CH(CH₃)CH(CH₃)₂ | Cl | 2,6-F₂ |
| 85 | 3,5-dimethyl-1,2,4-triazol-1-yl | 4-methyl-piperidin-1-yl | Cl | 2-F, 6-Cl |
| 86 | 3-nitro-1,2,4-triazol-1-yl | 4-methyl-piperidin-1-yl | Cl | 2,6-F₂, 4-OCH₃ |
| 87 | pyrazol-1-yl | 4-methyl-piperidin-1-yl | Cl | 2-F, 4-CH₃ |
| 88 | 5-ethoxycarbonyl-3-methyl-pyrazol-1-yl | (R)-NHCH(CH₃)(CH(CH₃)₂) | Cl | 2,4,6-F₃ |
| 89 | 3-nitro-1,2,4-triazol-1-yl | (R)-NHCH(CH₃)(CH(CH₃)₂) | Cl | 2,4,6-F₃ |
| 90 | 1,2,3-triazol-1-yl | 4-methyl-piperidin-1-yl | CH₃ | 2,4,6-F₃ |
| 91 | 1,2,3-triazol-1-yl | NH—CH(CH₃)(C₂H₅) | Cl | 2,4,6-F₃ |
| 92 | 3-methyl-pyrazol-1-yl | (R)-NHCH(CH₃)(CH(CH₃)₂) | Cl | 2,4,6-F₃ |
| 93 | 1,2,4-triazol-1-yl | 4-methyl-piperidin-1-yl | CH₃ | 2,4,6-F₃ |
| 94 | 3-amino-1,2,4-triazol-1-yl | 4-methyl-piperidin-1-yl | Cl | 2,4,6-F₃ |
| 95 | 3-(furan-2-yl)-4-methylpyrazol-1-yl | NHCH(CH₃)(CF₃) | Cl | 2,4,6-F₃ |
| 96 | pyrazol-1-yl | 2-methyl-piperidin-1-yl | Cl | 2,4,6-F₃ |
| 97 | pyrazol-1-yl | NH—CH(CH₃)(C₂H₅) | Cl | 2-F, 4-CH₃ |
| 98 | 1,2,4-triazol-1-yl | 2-methyl-pyrrolidin-1-yl | Cl | 2-F, 6-Cl |
| 99 | pyrazol-1-yl | 3-methyl-piperidin-1-yl | Cl | 2-F, 4-CH₃ |
| 100 | 1,2,4-triazol-1-yl | (S)-NHCH(CH₃)(CH(CH₃)₂) | Cl | 2-F, 4-CH₃ |
| 101 | pyrazol-1-yl | NH—CH(CH₃)₂ | Cl | 2,4,6-F₃ |
| 102 | pyrazol-1-yl | (S)-NHCH(CH₃)(C₂H₅) | Cl | 2-F, 4-CH₃ |
| 103 | pyrazol-1-yl | NH—CH₂CH₂CH₃ | Cl | 2-F, 4-CH₃ |
| 104 | 3-amino-pyrazol-1-yl | NH—CH(CH₃)₂ | Cl | 2,4,6-F₃ |
| 105 | pyrazol-1-yl | NH—CH(CH₃)(C₂H₅) | Cl | 2,4-F₂ |
| 106 | pyrazol-1-yl | NH—CH(CH₃)(C₂H₅) | Cl | 2-F, 6-Cl |
| 107 | 1,2,3-triazol-1-yl | NH—CH(CH₃)(C₂H₅) | Cl | 2-F, 6-Cl |
| 108 | pyrazol-1-yl | NH—CH₂CF₃ | Cl | 2-F, 4-CH₃ |
| 109 | pyrazol-1-yl | NH—CH(CH₃)(C₂H₅) | Cl | 2-F, 6-CH₃ |
| 110 | 1,2,4-triazol-1-yl | NH—CH(CH₃)(C₂H₅) | Cl | 2-F, 6-CH₃ |
| 111 | 1,2,3-triazol-1-yl | NH—CH(CH₃)(C₂H₅) | Cl | 2-F, 6-CH₃ |
| 112 | —ON═C(CH₃)₂ | NH—CH(CH₃)(C₂H₅) | Cl | 2-F, 6-CH₃ |
| 113 | 1,2,4-triazol-1-yl | NH—CH(CH₃)(C₂H₅) | Cl | 2,6-F₂ |
| 114 | 1,2,3-triazol-1-yl | NH—CH(CH₃)(C₂H₅) | Cl | 2,6-F₂ |
| 115 | pyrazol-1-yl | 4-methyl-piperidin-1-yl | Cl | 2,6-F₂ |
| 116 | 1,2,4-triazol-1-yl | 4-methyl-piperidin-1-yl | Cl | 2,6-F₂ |
| 117 | 1,2,3-triazol-1-yl | 4-methyl-piperidin-1-yl | Cl | 2,6-F₂ |
| 118 | 3,5-dimethyl-1,2,4-triazol-1-yl | 4-methyl-piperidin-1-yl | Cl | 2,6-F₂ |
| 119 | 1,2,3-triazol-1-yl | 4-methyl-piperidin-1-yl | Cl | 2-Cl, 4-F |
| 120 | 4-iodo-pyrazol-1-yl | NH—CH(CH₃)(C₂H₅) | Cl | 2-F, 6-CH₃ |
| 121 | 3-amino-pyrazol-1-yl | NH—CH(CH₃)(CH₂CH₂CH₃) | Cl | 2-F, 4-CH₃ |
| 122 | 3-amino-pyrazol-1-yl | NH—CH₂C(CH₃)═CH₂ | Cl | 2,4,6-F₃ |
| 123 | 4-bromo-pyrazol-1-yl | N(CH₃)—CH₂CH═CH₂ | Cl | 2,4,6-F₃ |
| 124 | 4-bromo-pyrazol-1-yl | NH—CH(CH₃)CH₂OH | Cl | 2,4,6-F₃ |
| 125 | pyrazol-1-yl | 2-methyl-piperidin-1-yl | Cl | 2,6-F₂ |
| 126 | 1,2,3-triazol-1-yl | 2-methyl-piperidin-1-yl | Cl | 2,6-F₂ |
| 127 | 3-amino-pyrazol-1-yl | 2-methyl-piperidin-1-yl | Cl | 2,6-F₂ |
| 128 | 3-amino-pyrazol-1-yl | NH—CH(CH₃)CH₂OCH₃ | Cl | 2-F, 4-CH₃ |
| 129 | thiazol-2-yl | (S)-NHCH(CH₃)(CF₃) | Cl | 2,4,6-F₃ |
| 130 | -C(NH₂)═NOCH₃ | (R)-NHCH(CH₃)(CF₃) | Cl | 2,4,6-F₃ |
| 131 | 3-amino-pyrazol-1-yl | N(CH₃)—CH₂CH₂CH═CH₂ | Cl | 2-F, 6-Cl |
| 132 | pyrazol-1-yl | N(CH₃)—CH₂CH₂CH═CH₂ | Cl | 2-Cl, 4-F |
| 133 | 4-methyl-pyrazol-1-yl | N(CH₃)—CH₂CH₂CH═CH₂ | Cl | 2-Cl, 4-F |
| 134 | 4-bromo-pyrazol-1-yl | N(CH₂CH═CH₂)₂ | Cl | 2-Cl, 4-F |
| 135 | 3-amino-pyrazol-1-yl | N(CH₂CH═CH₂)₂ | Cl | 2-Cl, 4-F |
| 136 | thiazol-2-yl | (S)-NH—CH(CH₃)CH(CH₃)₂ | Cl | 2-F, 6-Cl |
| 137 | —C(NH₂)═NOH | (R)-NHCH(CH₃)(CF₃) | Cl | 2,4,6-F₃ |
| 138 | pyrazol-1-yl | (S)-NH—CH(CH₃)CH(CH₃)₂ | Cl | 2,4,6-F₃ |
| 139 | 1,2,3-triazol-1-yl | (S)-NH—CH(CH₃)CH(CH₃)₂ | Cl | 2,4,6-F₃ |
| 140 | pyrazol-1-yl | 2-methyl-pyrrolidin-1-yl | Cl | 2,6-F₂ |
| 141 | 1,2,4-triazol-1-yl | 2-methyl-piperidin-1-yl | Cl | 2,4-F₂ |
| 142 | pyrazol-1-yl | N(CH₃)—CH₂CH═CH₂ | Cl | 2,4,6-F₃ |
| 143 | 3-amino-pyrazol-1-yl | NH—CH(CH₃)C₂H₅ | Cl | 2-F, 6-CH₃ |
| 144 | —C(NH₂)═NOH | NH—CH(CH₃)₂ | Cl | 2,4,6-F₃ |
| 145 | —C(NH₂)═NOH | (S)-NH—CH(CH₃)CH(CH₃)₂ | Cl | 2,4,6-F₃ |
| 146 | —C(NH₂)═NOH | NH—CH(CH₃)C₂H₅ | Cl | 2,4,6-F₃ |
| 147 | —C(NH₂)═NOCH₃ | (S)-NH—CH(CH₃)CH(CH₃)₂ | Cl | 2,4,6-F₃ |
| 148 | 3-amino-pyrazol-1-yl | NH—CH(CH₃)(C₂H₅) | Cl | 2-F, 6-Cl |
| 149 | 3-amino-pyrazol-1-yl | NH—CH₂CF₃ | Cl | 2-F, 4-CH₃ |
| 150 | 4-chloro-pyrazol-1-yl | NH—CH₂CF₃ | Cl | 2-F, 4-CH₃ |
| 151 | 3-benzylsulfanyl-1,2,4-triazol-1-yl | (S)-NHCH(CH₃)(CF₃) | Cl | 2,4,6-F₃ |
| 152 | —NHN═CH(CH(CH₃)C(O)OC₂H₅) | (S)-NHCH(CH₃)(CF₃) | Cl | 2,4,6-F₃ |
| 153 | 4-methyl-5-oxo-2,5-dihydro-pyrazol-1-yl | (S)-NHCH(CH₃)(CF₃) | Cl | 2,4,6-F₃ |
| 154 | 5-methoxy-4-methyl-pyrazol-1-yl | (S)-NHCH(CH₃)(CF₃) | Cl | 2,4,6-F₃ |
| 155 | 5-chloro-4-methyl-pyrazol-1-yl | (S)-NHCH(CH₃)(CF₃) | Cl | 2,4,6-F₃ |
| 156 | pyrazol-1-yl | (S)-NHCH(CH₃)(CF₃) | CH₃ | 2,4,6-F₃ |
| 157 | 1,2,3-triazol-1-yl | (S)-NHCH(CH₃)(CF₃) | CH₃ | 2,4,6-F₃ |
| 158 | —C(NH₂)═NOC₂H₅ | (R)-NHCH(CH₃)(CF₃) | Cl | 2,4,6-F₃ |
| 159 | —C(O)NH₂ | (S)-NHCH(CH₃)(CF₃) | Cl | 2,4,6-F₃ |
| 160 | 5-ethoxycarbonyl-3-methyl-pyrazol-1-yl | NH—CH₂CH₂CH₃ | Cl | 2-F, 4-CH₃ |
| 161 | pyrazol-1-yl | 2-methyl-piperidin-1-yl | Br | 2,4,6-F₃ |
| 162 | 4-cyano-pyrazol-1-yl | (S)-NHCH(CH₃)(CF₃) | Cl | 2,4,6-F₃ |
| 163 | 4-cyano-pyrazol-1-yl | NH—CH(CH₃)C₂H₅ | Cl | 2-F, 6-Cl |
| 164 | pyrazol-1-yl | NH—C₂H₅ | Cl | 2,4,6-F₃ |
| 165 | 1,2,3-triazol-2-yl | (S)-NHCH(CH₃)(CF₃) | Br | 2,4,6-F₃ |
| 166 | 1,2,3-triazol-1-yl | 4-methyl-piperidin-1-yl | CH₃ | 2-F, 6-Cl |
| 167 | pyrazol-1-yl | (S)-NHCH(CH₃)(CF₃) | F | 2,4,6-F₃ |
| 168 | —C(NH₂)═NOH | (S)-NHCH(CH₃)(C₂H₅) | Cl | 2-Cl, 4-F |
| 169 | —C(S)NH₂ | (S)-NHCH(CH₃)(CF₃) | Cl | 2-F, 6-Cl |
| 170 | —C(NH₂)═NOCH₃ | 2-methyl-pyrrolidinyl-1-yl | Cl | 2-Cl, 4-F |
| 171 | —C(NH₂)═NOH | (S)-NHCH(CH₃)(CF₃) | CH₃ | 2,4,6-F₃ |
| 172 | —C(NH₂)═NOH | (S)-NHCH(CH₃)(CF₃) | Cl | 2-Cl, 4-F |
| 173 | —C(NH₂)═NOH | NH—CH₂CF₃ | Cl | 2,4,6-F₃ |
| 174 | —C(O)NH(CH₃) | (S)-NHCH(CH₃)(CF₃) | Cl | 2,4,6-F₃ |
| 175 | —C(NH₂)═NOH | (S)-NHCH(CH₃)(CF₃) | Cl | 2,6-F₂ |
| 176 | —C(NH₂)═NOH | (S)-NHCH(CH₃)(CF₃) | Cl | 2-F, 6-Cl |
| 177 | —C(NH₂)═NOCHF₂ | (S)-NHCH(CH₃)(CF₃) | Cl | 2,4,6-F₃ |
| 178 | 4-methyl-thiazol-2-yl | (S)-NHCH(CH₃)(CF₃) | Cl | 2,4,6-F₃ |
| 179 | —C(O)NH₂ | 4-methyl-piperidin-1-yl | Cl | 2,6-F₂ |
| 180 | —C(O)NH₂ | (S)-NHCH(CH₃)(CF₃) | Cl | 2,6-F₂ |
| 181 | —C(NH₂)═NOH | (S)-NHCH(CH₃)(CF₃) | Cl | 2,6-F₂, 4-OCH₃ |
| 182 | —C(NH₂)═NOCH₃ | (S)-NHCH(CH₃)(CF₃) | Cl | 2,6-F₂, 4-OCH₃ |
| 183 | —C(O)NH₂ | (S)-NHCH(CH₃)CH(CH₃)₂ | Cl | 2-Cl, 4-OCH₃ |
| 184 | —C(O)NHC(O)CH₃ | 4-methyl-piperidin-1-yl | Cl | 2,6-F₂ |
| 185 | —C(NH₂)═NOH | (S)-NH—CH(CH₃)CH(CH₃)₂ | Cl | 2-Cl, 4-OCH₃ |
| 186 | —C(NH₂)═NOCH₃ | (S)-NH—CH(CH₃)CH(CH₃)₂ | Cl | 2-Cl, 4-OCH₃ |
| 187 | 3-amino-4-cyano-pyrazol-1-yl | (S)-NHCH(CH₃)(CF₃) | Cl | 2-F, 6-Cl |
| 188 | —C(O)NH₂ | 4-methyl-piperidin-1-yl | Cl | 2,6-F₂, 4-OCH₃ |
| 189 | —C(O)NH₂ | (S)-NHCH(CH₃)CF₃) | Cl | 2,6-F₂, 4-OCH₃ |
| 190 | —C(NH₂)═NOH | 4-methyl-piperidin-1-yl | Cl | 2,6-F₂, 4-OCH₃ |
| 191 | —C(NH₂)═NOH | (S)-NH—CH(CH₃)CH(CH₃)₂ | Cl | 2,6-F₂, 4-OCH₃ |
| 192 | —C(NH₂)═NOH | (S)-NH—CH(CH₃)CH(CH₃)₂ | Cl | 2-Cl, 4-NO₂ |
| 193 | —C(NH₂)═NOH | (S)-NH—CH(CH₃)CH(CH₃)₂ | Cl | 2-Cl, 4-F |
| 194 | —C(NH₂)═NOH | 4-methyl-piperidin-1-yl | Cl | 2,6-F₂ |
| 195 | —C(NH₂)═NOH | (S)-NH—CH(CH₃)CH(CH₃)₂ | Cl | 2,6-F₂ |
| 196 | —C(NH₂)═NOCH₃ | (S)-NH—CH(CH₃)CH(CH₃)₂ | Cl | 2,6-F₂ |
| 197 | —C(NH₂)═NOCH₃ | 4-methyl-piperidin-1-yl | Cl | 2,6-F₂, 4-OCH₃ |
| 198 | —C(NH₂)═NOCH₃ | (S)-NH—CH(CH₃)CH(CH₃)₂ | Cl | 2,6-F₂, 4-OCH₃ |
| 199 | —C(O)NH₂ | (S)-NH—CH(CH₃)CH(CH₃)₂ | Cl | 2,6-F₂, 4-OCH₃ |
| 200 | —C(CH₃)═NOH | (S)-NH—CH(CH₃)CH(CH₃)₂ | Cl | 2-Cl, 4-OCH₃ |
| 201 | —C(NH₂)═NOH | (S)-NH—CH(CH₃)CH(CH₃)₂ | Cl | 2-Cl, 5-F |
| 202 | —C(NH₂)═NOCH₃ | (S)-NH—CH(CH₃)CH(CH₃)₂ | Cl | 2-Cl, 5-F |
| 203 | —C(S)NH₂ | (S)-NHCH(CH₃)(CF₃) | Cl | 2,6-F₂, 4-OCH₃ |
| 204 | —ON═C(CH₃)₂ | (S)-NHCH(CH₃)(CF₃) | Cl | 2,4,6-F₃ |
| 205 | 1,2,3-triazol-1-yl | (S)-NHCH(CH₃)(CF₃) | Cl | 2,4,6-F₃ |
| 206 | 1,2,3-triazol-1-yl | N(CH₃)(CH₂CH═CH₂) | Cl | 2,4,6-F₃ |
| 207 | pyrazol-1-yl | (S)-NHCH(CH₃)(CF₃) | Br | 2,4,6-F₃ |
| 208 | —C(NH₂)═NOH | 2-methyl-pyrrolidin-1-yl | Cl | 2-Cl, 4-F |
| 209 | —C(CH₃)═NOH | (S)-NHCH(CH₃)(CF₃) | Cl | 2,4,6-F₃ |
| 210 | 2-oxo-pyrrolidin-1-yl | NHCH₂CF₃ | Cl | 2,4,6-F₃ |
| 211 | —C(NH₂)═NOCH₃ | (S)-NHCH(CH₃)(CF₃) | Cl | 2-Cl, 4-F |
| 212 | 1,2,3-triazol-1-yl | NHCH₂CF₃ | Cl | 2,4,6-F₃ |
| 213 | —C(NH₂)═NOCH₃ | (S)-NHCH(CH₃)(CF₃) | Cl | 2,6-F₂ |
| 214 | —C(NH₂)═NOCH₃ | (S)-NHCH(CH₃)(CF₃) | Cl | 2-F, 6-Cl |
| 215 | —C(NH₂)═NOH | (S)-NHCH(CH₃)(CF₃) | Cl | 2-Cl, 4-OCH₃ |
| 216 | —C(NH₂)═NOCH₃ | (S)-NHCH(CH₃)(CF₃) | Cl | 2-Cl, 4-OCH₃ |
| 217 | —C(O)NH₂ | (S)-NHCH(CH₃)(CF₃) | Cl | 2-Cl, 4-OCH₃ |
| 218 | —C(NH₂)═NOCH₃ | (S)-NH—CH(CH₃)CH(CH₃)₂ | Cl | 2-Cl, 4-F |
| 219 | —C(NH₂)═NOCH₃ | (S)-NH—CH(CH₃)CH(CH₃)₂ | Cl | 2-Cl, 4-NO₂ |
| 220 | —C(NH₂)═NOH | (S)-NHCH(CH₃)(CF₃) | Cl | 2-Cl, 5-F |
| 221 | —C(NH₂)═NOCH₃ | (S)-NHCH(CH₃)(CF₃) | Cl | 2-Cl, 5-F |

### Measurement of the Cell Cycle Inhibition in HeLa Cells—Test Procedure:

HeLa B cells are grown in DMEM (Life Technologies Cat No 21969-035) supplemented with 10% Fetal Calf Serum (FCS, Life Technologies Cat No 10270-106) in 180 cm² Flasks at 37° C., 92% humidity and 7% CO₂.

Cells are seeded at 5×10⁴ cells per well in a 24-well plate. Twenty hours later the compounds are added such that the final concentration is 1×10⁻⁶, 3.3×10⁻⁷, 1.1×10⁻⁷, 3.7×10⁻⁸, 1.2×10⁻⁸ and 1×10⁻⁹ M in a final volume of 500 μl. DMSO alone is added to 6 wells as a control. Cells are incubated with the compounds as above for 20 h. Then cells are observed under the microscope to check for cell death, and the 24-well plate is then centrifuged at 1200 rpm for 5 min at 20° C., acceleration position 7 and break position 5 (Eppendorf centrifuge 5804R).

The supernatant is removed and the cells lysed with 0.5 ml RNase Buffer (10 mM NaCitrate, 0.1% Nonidet NP40, 50 μg/ml RNase, 10 μg/ml Propidium iodide) per well. The plates are then incubated for at least 30 min in the dark at RT and the samples then transferred to FACS tubes. Samples are measured in a FACS machine (Beckton Dickinson) at the following settings:

### Instrument Settings of the FACS Calibur:

### Run Modus: High

| Parameter | Voltage | Amp Gain | Mode |
|---|---|---|---|
| FSC | E01 | 2.5 | lin |
| SSC | 350 | 1 | lin |
| FI 1 | | | |
| FI 2 | 430 | 2 | lin |
| FI 3 | | | |
| FI 2 - A | — | 1 | lin |
| FI 2 - W | — | 3 | lin |

| | |
|---|---|
| DDM Parameter | FI 2 |

The ratio of cells in G₀/G₁-phase to G₂/M phase is calculated and compared to the value for the controls (DMSO) only. Results are given in table 2 as the IC₅₀ value calculated from the concentration curve plotted against the cell cycle ratio and indicate the compound concentration at which 50% of cells are in cell cycle arrest after treatment with the compound.

Test on other cell lines (MCF-7 and COLO 205) were done in the same way except that they were incubated with the growth medium recommended by the American Tissue Culture collection for that cell type.

| Example | IC₅₀ [nM] |
|---|---|
| 1 | 4.8 |
| 2 | 48 |
| 3 | 31 |
| 4 | 41 |
| 5 | 4.6 |
| 6 | 17 |
| 7 | 21 |
| 8 | 13 |
| 9 | 13 |
| 10 | 47 |
| 11 | 42 |
| 12 | 6.9 |
| 13 | 16 |
| 14 | 14 |
| 15 | 43 |
| 16 | 46 |
| 17 | 45 |
| 18 | 39 |
| 19 | 16 |
| 20 | 39 |
| 21 | 25 |
| 22 | 32 |
| 23 | 39 |
| 24 | 50 |
| 25 | 24 |
| 26 | 38 |
| 27 | 3.5 |
| 28 | 17 |
| 29 | 17 |
| 30 | 48 |
| 31 | 49 |
| 32 | 43 |
| 33 | 11 |
| 34 | 25 |
| 35 | 36 |
| 36 | 7.4 |
| 37 | 32 |
| 38 | 24 |
| 39 | 26 |
| 40 | 23 |
| 41 | 38 |
| 42 | 18 |
| 43 | 19 |
| 44 | 18 |
| 45 | 17 |
| 46 | 38 |
| 47 | 26 |
| 48 | 13 |
| 49 | 10 |
| 50 | 9.1 |
| 51 | 6.5 |
| 52 | 22 |
| 53 | 26 |
| 54 | 23 |
| 55 | 26 |
| 56 | 11 |
| 57 | 5.8 |
| 58 | 26 |
| 59 | 43 |
| 60 | 19 |
| 61 | 21 |
| 62 | 23 |
| 63 | 22 |
| 64 | 21 |
| 65 | 20 |
| 66 | 37 |
| 67 | 13 |
| 68 | 20 |
| 69 | 21 |
| 70 | 35 |
| 71 | 25 |
| 72 | 46 |
| 73 | 11 |
| 74 | 13 |
| 75 | 14 |
| 76 | 7.6 |
| 77 | 35 |
| 78 | 21 |
| 79 | 21 |
| 80 | 26 |
| 81 | 34 |
| 82 | 30 |
| 83 | 37 |
| 84 | 27 |
| 85 | 21 |
| 86 | 24 |
| 87 | 39 |
| 88 | 44 |
| 89 | 47 |
| 90 | 27 |
| 91 | 20 |
| 92 | 26 |
| 93 | 39 |
| 94 | 25 |
| 95 | 39 |
| 96 | 29 |
| 97 | 13 |
| 98 | 46 |
| 99 | 39 |
| 100 | 40 |
| 101 | 33 |
| 102 | 50 |
| 103 | 39 |
| 104 | 47 |
| 105 | 45 |
| 106 | 12 |
| 107 | 39 |
| 108 | 16 |
| 109 | 25 |
| 110 | 25 |
| 111 | 29 |
| 112 | 21 |
| 113 | 49 |
| 114 | 41 |
| 115 | 23 |
| 116 | 42 |
| 117 | 19 |
| 118 | 32 |
| 119 | 48 |
| 120 | 25 |
| 121 | 50 |
| 122 | 46 |
| 123 | 49 |
| 124 | 45 |
| 125 | 38 |
| 126 | 38 |
| 127 | 37 |
| 128 | 38 |
| 129 | 14 |
| 130 | 1.8 |
| 131 | 48 |
| 132 | 46 |
| 133 | 41 |
| 134 | 50 |
| 135 | 18 |
| 136 | 29 |
| 137 | 1.5 |
| 138 | 23 |
| 139 | 26 |
| 140 | 20 |
| 141 | 46 |
| 142 | 39 |
| 143 | 32 |
| 144 | 25 |
| 145 | 23 |
| 146 | 32 |
| 147 | 41 |
| 148 | 34 |
| 149 | 41 |
| 150 | 50 |
| 151 | 8.3 |
| 152 | 24 |
| 153 | 27 |
| 154 | 26 |
| 155 | 22 |
| 156 | 15 |
| 157 | 19 |
| 158 | 44 |
| 159 | 23 |
| 160 | 31 |
| 161 | 50 |
| 162 | 17 |
| 163 | 30 |
| 164 | 48 |
| 165 | 30 |
| 166 | 42 |
| 167 | 20 |
| 168 | 36 |
| 169 | 41 |
| 170 | 59 |
| 171 | 54 |
| 172 | 21 |
| 173 | 18 |
| 174 | 42 |
| 175 | 18 |
| 176 | 20 |
| 177 | 21 |
| 178 | 20 |
| 179 | 53 |
| 180 | 41 |
| 181 | 6.0 |
| 182 | 11 |
| 183 | 53 |
| 184 | 51 |
| 185 | 30 |
| 186 | 33 |
| 187 | 39 |
| 188 | 30 |
| 189 | 30 |
| 190 | 26 |
| 191 | 12 |
| 192 | 30 |
| 193 | 9.0 |
| 194 | 21 |
| 195 | 20 |
| 196 | 38 |
| 197 | 42 |
| 198 | 15 |
| 199 | 33 |
| 200 | 47 |
| 201 | 30 |
| 202 | 38 |
| 203 | 47 |
| 204 | 23 |
| 205 | 8.3 |
| 206 | 20 |
| 207 | 15 |
| 208 | 56 |
| 209 | 18 |
| 210 | 39 |
| 211 | 24 |
| 212 | 53 |
| 213 | 51 |
| 214 | 18 |
| 215 | 14 |
| 216 | 27 |
| 217 | 23 |
| 218 | 29 |
| 219 | 29 |
| 220 | 36 |
| 221 | 30 |

1-14. (canceled)

15. A pharmaceutical composition for cancer therapy comprising a substituted 5-phenyl pyrimidine of formula (I) 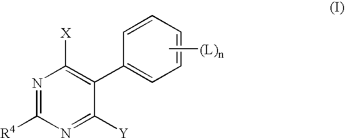
and/or pharmaceutically acceptable salts thereof; wherein
X is NR¹R², OR^{1a}, or SR^{1a}, wherein
R¹, R², and R^{1a}
are, independently of each other, hydrogen; C₁-C₁₀-alkyl; C₂-C₆-alkenyl; C₂-C₆-alkynyl; C₁-C₁₀-haloalkyl; C₃-C₈-cycloalkyl; C₃-C₈-halocycloalkyl; phenyl; or 5- or 6-membered heteroaryl or 5- or 6-membered heterocyclyl, containing 1, 2, 3 or, 4 nitrogen atoms or 1, 2, or 3 nitrogen atoms and one sulfur or oxygen atom as ring members; wherein said alkyl, alkenyl, alkynyl, haloalkyl, cycloalkyl, halocycloalkyl, phenyl, heteroaryl, and heterocyclyl are optionally substituted with 1, 2, 3, or 4 radicals R^{a1}; and wherein NR¹R² optionally defines a 5- or 6-membered optionally substituted heterocyclic ring containing 1, 2, 3, or 4 nitrogen atoms or 1, 2, or 3 nitrogen atoms and one sulfur or oxygen atom as ring members, which are non-adjacent to the nitrogen of NR¹R², and wherein two adjacent C atoms or one N atom and one adjacent C atom are optionally linked by a C₁-C₄-alkylene chain and wherein said heterocyclic ring is optionally substituted with 1, 2, 3, or 4 radicals R^{a1}; wherein
R^{a1} is halogen; oxo; nitro; cyano; hydroxy; C₁-C₆-alkyl; C₃-C₆-cycloalkyl; C₃-C₆-cycloalkenyl; C₁-C₆-haloalkyl; C₁-C₆-alkoxy; C₁-C₆-alkylthio; —C(═O)-A; —C(═O)—O-A; —C(O)—N(A′)A; C(A′)(═N-OA); N(A′)A; N(A′)-C(═O)-A; N(A″)-C(═O)—N(A′)A; S(═O)ₘ-A, S(═O)ₘ—O-A; S(═O)ₘ—N(A′)A; phenyl; or 5- or 6-membered heteroaryl, containing 1, 2, 3, or 4 nitrogen atoms as ring members or 1, 2 or 3 nitrogen atoms and one sulfur or oxygen atom as ring members; wherein said phenyl and said heteroaryl is optionally substituted with one to three radicals selected from the group consisting of halogen, C₁-C₆-alkyl, C₂-C₆-alkenyl, C₂-C₆-alkynyl, C₃-C₆-cycloalkyl, C₃-C₆-halogenalkyl, C₁-C₆-alkoxy, cyano, nitro, —C(═O)-A, —C(═O)—O-A, —C(═O)—N(A′)A, C(A′)(═N-OA), and N(A′)A; wherein
m is 0, 1, or 2; and
A, A′, and A″
are, independently of each other, hydrogen; C₁-C₆-alkyl; C₂-C₆-alkenyl; C₂-C₆-alkynyl; C₃-C₈-cycloalkyl; C₃-C₈-cycloalkenyl; or phenyl; wherein said alkyl, alkenyl, alkynyl, cycloalkyl, cycloalkenyl, and phenyl are optionally partially or fully halogenated or are optionally substituted by nitro, cyanato, cyano, or C₁-C₄-alkoxy; or A and A′ together with the atoms to which they are attached are a five- or six-membered saturated, partially unsaturated, or aromatic heterocycle which contains one to four heteroatoms selected from the group consisting of O, N, and S;
with the proviso that R^{1a} is not hydrogen;
Y is selected from the group consisting of halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino, or C₁-C₆-alkylamino, wherein said alkyl, alkenyl, and alkynyl are optionally substituted by halogen, cyano, nitro, C₁-C₂-alkoxy, or C₁-C₄-alkoxycarbonyl;
L is a radical comprising up to 10 atoms and which is selected from the group consisting of carbon, halogen, nitrogen, oxygen, and sulfur, wherein L comprises from 0 to 10 carbon atoms, from 0 to 5 halogen atoms, and from 0 to 4 heteroatoms different from halogen, and wherein L is not hydrogen;
n is 0, 1, 2, 3, 4, or 5;
R⁴ is a radical comprising from 1 to 15 non-hydrogen atoms and which are selected from the group consisting of carbon, halogen, nitrogen, oxygen, and sulfur, wherein R⁴ comprises from 0 to 10 carbon atoms, from 0 to 5 halogen atoms, and from 1 to 4 heteroatoms different from halogen, wherein R⁴ is not hydrogen, and wherein R⁴ is selected from the group consisting of R^{4a}, R^{4b}, R^{4c}, and R^{4d}, wherein
R^{4a} is cyano, hydroxy, mercapto, N₃, C₁-C₆-alkyl, C₂-C₈-alkenyl, C₂-C₈-alkynyl, C₁-C₆-haloalkyl, C₁-C₆-alkoxy, C₃-C₈-alkenyloxy, C₃-C₈-alkynyloxy, C₁-C₆-haloalkoxy, C₁-C₆-alkylthio, C₃-C₈-alkenylthio, C₃-C₈-alkynylthio, C₁-C₆-haloalkylthio, ON═CR^{a}R^{b}, —CR^{c}═NOR^{a}, —NR^{c}N═CR^{a}R^{b}, —NR^{c}NR^{a}R^{b}, —NOR^{a}, —NR^{c}C(—NR^{d})—NR^{a}R^{b}, NR^{c}C(═O)—NR^{a}R^{b}, NR^{a}C(═O)R^{c}, —NR^{a}C(═NOR^{c})—R^{d}, —O(C═O)R^{c}, —C(O)—OR^{a}, —C(O)—NR^{a}R^{b}, —C(═NOR^{c})—, —NR^{a}R^{b}, or —CR^{c}(═NNR^{a}R^{b}), wherein
R^{a}, R^{b}, R^{c}, and R^{d}
are, independently of each other, hydrogen; C₁-C₆-alkyl; C₂-C₈-alkenyl; C₂-C₈-alkynyl; C₁-C₆-haloalkyl; C₁-C₆-alkoxy; C₁-C₆-haloalkoxy; C₃-C₁₀-cycloalkyl, phenyl, five- to ten-membered saturated, partially unsaturated or aromatic mono- or bicyclic heterocycles comprising 1, 2, 3 or 4 heteroatoms selected from the group consisting of O, N, and S; wherein said C₁-C₆-alkyl, C₃-C₁₀-cycloalkyl, phenyl, and five- to ten-membered saturated, partially unsaturated or aromatic mono- or bicyclic heterocycles are optionally partially or fully halogenated or are optionally substituted with 1, 2, or 3 identical or different radicals R^{x}, wherein R^{a} is optionally C₁-C₆-alkylcarbonyl, and wherein R^{a} and R^{b} together optionally define a C₂-C₄-alkylene group which is optionally interrupted by an oxygen atom and/or comprises a double bond or R^{a} and R^{c} together optionally define a C₂-C₄-alkylene group which is optionally interrupted by an oxygen atom and/or comprises a double bond; wherein
R^{x} is cyano, nitro, amino, aminocarbonyl, aminothiocarbonyl, hydroxy, C₁-C₆-alkyl, C₁-C₆-haloalkyl, C₁-C₆-alkylcarbonyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heteroaryl, 5- or 6-membered heterocyclyl, 5- or 6-membered heteroaryloxy, C(═-NOR^{α})—OR^{β}, or OC(R^{α})₂—C(R^{β})═NOR^{β}, wherein said heteroaryl, heterocyclyl, and heteroaryloxy are optionally substituted by 1, 2, or 3 radicals R^{y}, wherein
R^{y} is cyano, nitro, halogen, hydroxy, amino, aminocarbonyl, aminothiocarbonyl, C₁-C₆-allyl, C₁-C₆-haloalkyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heteroaryl, 5- or 6-membered heterocyclyl, 5- or 6-membered heteroaryloxy, or C(NOR^{α})—OR^{β}; and wherein
R^{α} and R^{β} are hydrogen or C₁-C₆-alkyl;
R^{4b} is a 5 or 6-membered aromatic heterocyclic radical which comprises 1, 2, or 3 nitrogen atoms as ring members or 1 or 2 nitrogen atoms and 1 oxygen atom or sulfur atom as ring members, wherein R^{4b} is optionally substituted by one to three identical or different groups R⁴⁴, wherein
R⁴⁴ is halogen, hydroxyl, cyano, oxo, nitro, amino, mercapto, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, carboxyl, C₁-C₆-alkoxycarbonyl, carbamoyl, C₁-C₆-alkylaminocarbonyl, C₁-C₆-alkyl-C₁-C₆-alkylamincarbonyl, morpholinocarbonyl, pyrrolidinocarbonyl, C₁-C₆-alkylcarbonylamino, C₁-C₆-alkylamino, di(C₁-C₆-alkyl)amino, C₁-C₆-alkylthio, C₁-C₆-alkylsulfinyl, C₁-C₆-alkylsulfonyl, hydroxysulfonyl, aminosulfonyl, C₁-C₆-alkylaminosulfonyl, di(C₁-C₆-alkyl)aminosulfonyl, phenyl, or 5- or 6-membered heteroaryl comprising one to four hetero atoms selected from the group consisting of O, N, and S, wherein said alkyl, phenyl, heteroaryl, cycloalkyl, and alkoxy groups are optionally partially or fully halogenated or optionally substituted by 1, 2, or 3 identical or different radicals R^{x} as defined above;
R^{4c} is of the formulae (II) or (III) 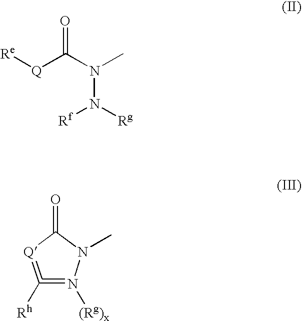
wherein
x is 0 or 1;
R^{e}, R^{f}, R^{g}, and R^{e#}
are, independently of one another, hydrogen, C₁-C₆-alkyl, C₂-C₈-alkenyl, C₂-C₈-alkynyl, C₃-C₆-cycloalkyl, C₄-C₆-cycloalkenyl, wherein said alkyl, alkenyl, alkynyl, cycloalkyl, cycloalkenyl are optionally partially or fully halogenated or are optionally substituted with one to four groups R^{v}, and wherein R^{f} and R^{g} together with the nitrogen atom to which they are attached optionally is R^{e}-Z-C(R^{h})═N;
Q is oxygen or N—R^{e#};
Q′ is C(H)—R^{k}, C—R^{k}, N—N(H)—R^{e#}, or N—R^{e#}; [symbol]
is a double bond or a single bond; wherein
R^{h} and R^{k}
are, independently of one another, hydrogen, halogen, cyano, C₁-C₆-alkyl, C₂-C₈-alkenyl, C₂-C₈-alkynyl, C₃-C₆-cycloalkyl, C₄-C₆-cycloalkenyl, wherein said alkyl, alkenyl, alkynyl, cycloalkyl, cycloalkenyl are optionally partially or fully halogenated or are optionally substituted with one to four groups and wherein R^{v} or R^{h} together with the carbon to which it is attached is optionally a carbonyl group; wherein
R^{v} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, and wherein two of R^{f}, R^{g}, R^{e}, or R^{e#} together with the atoms to which they are attached optionally define a five- or six-membered saturated, partially unsaturated, or aromatic heterocycle which contains one to four heteroatoms selected from the group consisting of O, N, and S;
R^{4d} is of the formulae (IV) or (V) 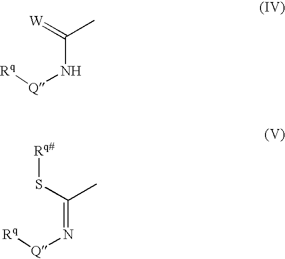
wherein
Q″ is a direct bond, —(C═O)—, —(C═O)—NH, —(C═O)—O—, —O—, or —NR^{p}—, wherein the molecule moiety to the left in each case is attached to the nitrogen atom;
R^{p} is hydrogen, methyl, or C₁-C₄-acyl;
R^{q} is hydrogen, methyl, benzyl, trifluoromethyl, allyl, propargyl, or methoxymethyl;
R^{q#} is hydrogen, C₁-C₆-alkyl; C₂-C₆-alkynyl;
W is S or NR^{q#};
wherein the aliphatic groups of R^{p}, R^{q}, and/or R^{q#} are optionally substituted with one or two groups R^{w}:
R^{w} is halogen, OR^{z}, NHR^{z}, C₁-C₆-alkyl, C₁-C₄-alkoxycarbonyl, C₁-C₄-acyl-amino, [1,3]dioxolane-C₁-C₄-alkyl, [1,3]dioxane-C₁-C₄-alkyl, wherein
R^{z} is hydrogen, methyl, allyl, or propargyl; and
a pharmaceutically acceptable carrier.

16. The pharmaceutical composition of claim 15, wherein R⁴ is R^{4a}.

17. The pharmaceutical composition of claim 15, wherein R⁴ is cyano, —ON═C^{a}R^{b}, CR^{c}═NOR^{a}, NR^{c}N═CR^{a}R^{b}, —NR^{c}NR^{a}R^{b}, —NR^{c}C(O)—NR^{a}R^{b}, —NR^{a}C(═O)R^{c}, NR^{a}C(═NOR^{c})—R^{d}, —C(O)—NR^{a}R^{b}, —C(═NOR^{c})—NR^{a}R^{b}, or —CR^{c}(═NNR^{a}R^{b}), wherein R^{a}, R^{b}, R^{c}, R^{d} are, independently of each other, hydrogen, C₁-C₆-alkyl, C₂-C₈-alkenyl, C₂-C₈-alkynyl, C₁-C₆-haloalkyl, C₁-C₆-alkoxy, or C₁-C₆-haloalkoxy, wherein R^{a} is optionally C₁-C₆-alkylcarbonyl, and wherein R^{a} and R^{b} together optionally define a C₂-C₄-alkylene group which is optionally interrupted by an oxygen atom and/or comprises a double bond or R^{a} and R^{c} together optionally define a C₂-C₄-alkylene group which is optionally interrupted by an oxygen atom and/or comprises a double bond.

18. The pharmaceutical composition of claim 15, wherein R⁴ is R^{4b}.

19. The pharmaceutical composition of claim 15, wherein R⁴ is R^{4c}.

20. The pharmaceutical composition of claim 15, wherein R⁴ is R^{4d}.

21. The pharmaceutical composition of claim 15, wherein said substituted 5-phenyl pyrimidines are of formula (Ia) 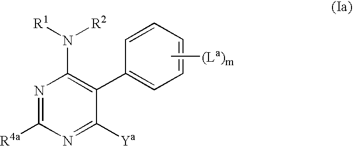
and/or pharmaceutically acceptable salts thereof; wherein
m is 1, 2, 3, 4, or 5;
Y^{a} is halogen, cyano, C₁-C₆-alkyl, C₁-C₆-haloalkyl, C₁-C₆-alkoxy, C₁-C₄-haloalkoxy, or C₃-C₆-alkenyloxy; and
L^{a} is, independently of each other, halogen, C₁-C₆-alkyl, C₁-C₆-alkoxy, or C₁-C₆-haloalkyl.

22. The pharmaceutical composition of claim 15, wherein said substituted 5-phenyl pyrimidines are of formula (Ib) 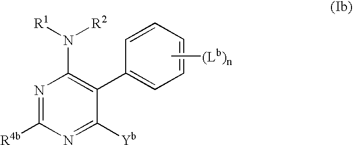
and/or pharmaceutically acceptable salts thereof; wherein
n is 1, 2, 3, 4 or 5;
Y^{b} is halogen, cyano, C₁-C₆-alkyl, C₁-C₆-haloalkyl, C₁-C₆-alkoxy, C₁-C₄-haloalkoxy, or C₃-C₆-alkenyloxy; and
L^{b} is, independently of each other, halogen, C₁-C₆-alkyl, C₁-C₆-alkoxy, C₁-C₆-haloalkyl, C₁-C₆-haloalkoxy, C₃-C₆-cycloalkoxy, C₁-C₆-alkoxycarbonyl, or C₁-C₆-alkylaminocarbonyl.

23. The pharmaceutical composition of claim 15, wherein said substituted 5-phenyl pyrimidines are of formula (Ic) 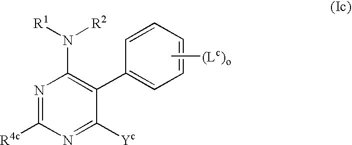
and/or pharmaceutically acceptable salts thereof; wherein
o is 1, 2, 3, 4 or 5
Y^{c} is halogen, cyano, C₁-C₄-alkyl, C₂-C₄-alkenyl, C₂-C₄-alkynyl, C₁-C₄-alkoxy, C₃-C₄-alkenyloxy, or C₃-C₄-alkynyloxy, wherein said alkyl, alkenyl, and alkynyl are optionally substituted by halogen, cyano, nitro, C₁-C₂-alkoxy, or C₁-C₄-alkoxycarbonyl;
L^{c} is halogen, cyano, cyanato (OCN), C₁-C₈-alkyl, C₂-C₁₀-alkenyl, C₂-C₁₀-alkynyl, C₁-C₆-alkoxy, —C(═O)-A¹, —C(═O)—O-A¹, —C(═O)—N(A²)A¹, C(A²)(═N-OA¹), N(A²)A¹, N(A²)—C(O)-A¹, N(A³)-C(═O)—N(A²)A¹, S(O)ₚ-A¹, S(═O)ₚ—O-A¹, or S(═O)ₚ—N(A²)A¹, wherein
p is 0, 1 or 2; and
A¹, A², and A³
are, independently of one another, hydrogen, C₁-C₆-alkyl, C₂-C₆-alkenyl, C₂-C₆-alkynyl, C₃-C₈-cycloalkyl, C₃-C₈-cycloalkenyl, or phenyl, wherein said alkyl, alkenyl, alkynyl, cycloalkyl, cycloalkenyl, and phenyl are optionally partially or fully halogenated or are optionally substituted by cyano or C₁-C₄-alkoxy; or wherein A¹ and A² together with the atoms to which they are attached optionally define a five- or six-membered saturated, partially unsaturated, or aromatic heterocycle which contains one to four heteroatoms selected from the group consisting of O, N, and S; and where the aliphatic, alicyclic, or aromatic groups of L^{c} are optionally partially or fully halogenated or are optionally substituted with one to four groups R^{u}, wherein
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(O)ₚ-A¹, S(O)ₚ—O-A¹, or S(═O)ₚ—N(A²)A¹, wherein p, A¹, A², and A³ are as defined above and wherein the aliphatic, alicyclic or aromatic groups are optionally partially or fully halogenated or are optionally substituted with one to three groups R^{ua}, wherein R^{ua} is as defined as R^{u}.

24. The pharmaceutical composition of claim 15, wherein said substituted 5-phenyl pyrimidines are of formula (Id) 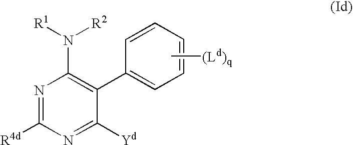
and/or pharmaceutically acceptable salts thereof; wherein
q is 1, 2, 3, 4 or 5
Y^{d} is halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino, or C₁-C₆-alkylamino, wherein said alkyl, alkenyl, and alkynyl are optionally substituted by halogen, cyano, nitro, C₁-C₂-alkoxy, or C₁-C₄-alkoxycarbonyl;
L^{d} is halogen, cyano, cyanato (OCN), C₁-C₈-alkyl, C₂-C₁₀-alkenyl, C₂-C₁₀-alkynyl, C₁-C₆-alkoxy, C₂-C₈-alkyenyloxy, C₂-C₈-alkynyloxy, C₃-C₆-cycloalkyl, C₄-C₆-cycloalkenyl, C₃-C₆-cycloalkyloxy, C₄-C₆-cycloalkenyloxy, nitro, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(═O)ₚ-A¹, S(O)ₚ—O-A¹ or S(═O)ₚ—N(A²)A¹, wherein
p is 0, 1, or 2; and
A¹, A², and A³
are, independently of one another, hydrogen, C₁-C₆-alkyl, C₂-C₆-alkenyl, C₂-C₆-alkynyl, C₃-C₈-cycloalkyl, C₃-C₈-cycloalkenyl, phenyl, wherein said alkyl, alkenyl, alkynyl, cycloalkyl, cycloalkenyl, and phenyl are optionally partially or fully halogenated or are optionally substituted by cyano or C₁-C₄-alkoxy; or wherein A¹ and A² together with the atoms to which they are attached optionally define a five- or six-membered saturated, partially unsaturated, or aromatic heterocycle which contains one to four heteroatoms selected from the group consisting of O, N and S; wherein the aliphatic, alicyclic, or aromatic groups of L^{d} are optionally partially or fully halogenated or are optionally substituted with one to four groups R^{u}:
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁-alkynyloxy, C₃-C₆-cycloalkyl, C₃-C₆-cycloalkenyl, C₃-C₆-cycloalkoxy, C₃-C₆-cycloalkenyloxy, —C(═O)-A¹, —C(═O)—O-A¹, —C(—O)—N(A²)A¹, C(A²)(═N-OA¹), N(A²)A¹, N(A²)-C(═O)-A¹, N(A³)-C(═O)—N(A²)A¹, S(═O)ₚ-A¹, S(═O)ₚ—O-A¹, or S(═O)ₚ—N(A²)A¹, wherein p, A¹, A², and A³ are as defined above and wherein the aliphatic, alicyclic or aromatic groups are optionally partially or fully halogenated or are optionally substituted with one to three groups R^{ua}, wherein R^{ua} is as defined as R^{u}.

25. The pharmaceutical composition of claim 15, wherein said substituted 5-phenyl pyrimidines are of formula (Ie) 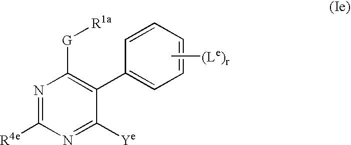
and/or pharmaceutically acceptable salts thereof; wherein
r is 1, 2, 3, 4 or 5;
Y^{e} is halogen, cyano, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the alkyl, alkenyl and alkynyl radicals of Y^{e} may be substituted by halogen, cyano, nitro, C₁-C₂-alkoxy or C₁-C₄-alkoxycarbonyl;
G is O or S;
L^{e} is halogen, cyano, cyanato (OCN), C₁-C₈-alkyl, C₂-C₁₀-alkenyl, C₂-C₁₀-alkynyl, C₁-C₆-alkoxy, C₂-C₈-alkyenyloxy, C₂-C₈-alkynyloxy, C₃-C₆-cycloalkyl, C₄-C₆-cycloalkenyl, C₃-C₆-cycloalkyloxy, C₄-C₆-cycloalkenyloxy, nitro, —C(═O)-A¹, —C(═O)—O-A¹, —C(═O)—N(A²)A¹, C(A)(═N-OA¹), N(A²)A¹, N(A²)-C(═O)-A¹, N(A³)-C(═O)—N(A²)A¹, S(═O)ₚ-A¹, S(═O)ₚ—O-A¹, or S(═O)ₚ—N(A²)A¹, wherein
p is 0, 1 or 2; and
A¹, A², and A³
are, independently of one another, hydrogen, C₁-C₆-alkyl, C₂-C₆-alkenyl, C₂-C₆-alkynyl, C₃-C₈-cycloalkyl, C₃-C₈-cycloalkenyl, or phenyl, wherein said alkyl, alkenyl, alkynyl, cycloalkyl, cycloalkenyl, and phenyl are optionally partially or fully halogenated or are optionally substituted by cyano or C₁-C₄-alkoxy; and wherein A¹ and A² together with the atoms to which they are attached optionally define a five- or six-membered saturated, partially unsaturated, or aromatic heterocycle which contains one to four heteroatoms selected from the group consisting of O, N, and S; wherein the aliphatic, alicyclic, or aromatic groups of L are optionally partially or fully halogenated or are optionally substituted with one to four groups R^{u}, wherein
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A¹, —C(═O)—O-A¹, —C(═O)—N(A²)A¹, C(A²)(═N-OA¹), N(A²)A¹, N(A²)-C(═O)-A¹, N(A³)-C(═O)—N(A²)A¹, S(═O)ₚ-A¹, S(═O)ₚ—O-A¹, or S(═O), —N(A²)A¹, wherein p, A¹, A², and A³ are as defined above and wherein the aliphatic, alicyclic or aromatic groups are optionally partially or fully halogenated or are optionally substituted with one to three groups R^{ua}, wherein R^{ua} is as defined as R^{u};
R^{4e} is a 5 or 6-membered aromatic heterocyclic radical which comprises 1, 2, or 3 nitrogen atoms as ring members or 1 or 2 nitrogen atoms and 1 oxygen atom or I sulfur atom as ring members, wherein R^{4e} is optionally substituted by one to three identical or different groups R⁴⁴, wherein
R⁴⁴ is halogen, hydroxyl, cyano, oxo, nitro, amino, mercapto, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, carboxyl, C₁-C₆-alkoxycarbonyl, carbamoyl, C₁-C₆-alkylaminocarbonyl, C₁-C₆-alkyl-C₁-C₆-alkylamincarbonyl, morpholinocarbonyl, pyrrolidinocarbonyl, C₁-C₆-alkylcarbonylamino, C₁-C₆-alkylamino, di(C₁-C₆-alkyl)amino, C₁-C₆-alkylthio, C₁-C₆-alkylsulfinyl, C₁-C₆-alkylsulfonyl, hydroxysulfonyl, aminosulfonyl, C₁-C₆-alkylaminosulfonyl, di(C₁-C₆-alkyl)aminosulfonyl, phenyl, 5- or 6-membered heteroaryl comprising one to four hetero atoms selected from the group consisting of O, N, and S, wherein said alkyl, phenyl, heteroaryl, cycloalkyl, and alkoxy groups are optionally partially or fully halogenated or are optionally substituted by 1, 2, or 3 identical or different radicals R^{x}; or
R^{4e} is cyano, hydroxy, mercapto, N₃, C₁-C₆-alkyl, C₂-C₈-alkenyl, C₂-C₈-alkynyl, C₁-C₆-haloalkyl, C₁-C₆-alkoxy, C₃-C₈-alkenyloxy, C₃-C₈-alkinyloxy, C₁-C₆-haloalkoxy, C₁-C₆-alkylthio, C₃-C₈-alkenylthio, C₃-C₈-alkynylthio, C₁-C₆-haloalkylthio, —ON═CR^{a}R^{b}, —CR^{c}═NOR^{a}, NR^{c}N═CR^{b}, NR^{c}NR^{a}R^{b}, —NOR^{a}; —NR^{c}C(═NR^{d})—NR^{a}R^{b}, NR^{c}C(═O)—NR^{a}R^{b}, —NR^{a}C(═O)R^{c}, —NR^{a}C(═NOR^{c})—R^{d}, —O(C═O)R^{c}, —C(═O)—OR^{a}, —C(═O)—NR^{a}R^{b}, —C(NOR^{c})—NR^{a}R^{b}, or —CR^{c}(═NNR^{a}R^{b}), wherein
R^{a}, R^{b}, R^{c}, and R^{d}
are, independently of each other, hydrogen, C₁-C₆-alkyl, C₂-C₈-alkenyl, C₂-C₈-alkynyl, C₁-C₆-haloalkyl, C₁-C₆-alkoxy, C₁-C₆-haloalkoxy, or a cyclic radical selected from the group consisting of C₃-C₁₀-cycloalkyl, phenyl, and five- to ten-membered saturated, partially unsaturated, or aromatic mono- or bicyclic heterocycles comprising 1, 2, 3, or 4 heteroatoms selected from the group consisting of O, N and S, wherein R^{a} is optionally C₁-C₆-alkylcarbonyl, wherein R^{a} and R^{b} together define a C₂-C₄-alkylene group which is optionally interrupted by an oxygen atom and/or comprises a double bond or R^{a} and R^{b} together define a C₂-C₄-alkylene group which is optionally interrupted by an oxygen atom and/or comprises a double bond, and wherein said C₁-C₆-alkyl and said cyclic radical are optionally partially or fully halogenated or are optionally substituted by 1, 2, or 3 identical or different radicals R^{x}, wherein
R^{x} is cyano, nitro, amino, aminocarbonyl, aminothiocarbonyl, hydroxy, C₁-C₆-alkyl, C₁-C₆-haloalkyl, C₁-C₈-alkylcarbonyl, C₁₋C₆₋alkylsulfonyl, C₁-C₆-alkylsulfoxyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heteroaryl, 5- or 6-membered heterocyclyl, 5- or 6-membered heteroaryloxy, C(═NOR^{α})—OR^{β}, or OC(R^{α})₂—C(R^{β})—NOR^{β}, wherein the cyclic radicals R^{x} are optionally substituted by 1, 2, or 3 radicals R^{y}, wherein
R^{y} is cyano, nitro, halogen, hydroxy, amino, aminocarbonyl, aminothiocarbonyl, C₁-C₆-alkyl, C₁-C₆-haloalkyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heteroaryl, 5- or 6-membered heterocyclyl, 5- or 6-membered heteroaryloxy, or C(NOW)—OR; and wherein
R^{α} and R^{β} are hydrogen or C₁-C₆-alkyl.

26. A method for treating cancer in an animal comprising administering to a subject in need thereof a therapeutically effective amount of the pharmaceutical composition of claim 15.
